(19) United States
(12) Patent Application Publication
(10) Pub. No.: US 2026/0182856 A1
(43) Pub. Date: Jul. 2, 2026

(54) Scanning Techniques to Determine Sensor Placement

(71) Applicant: iRhythm Technologies, Inc., San Francisco, CA (US)

(72) Inventor: Monica C. Lin, San Francisco, CA (US)

(73) Assignee: iRhythm Technologies, Inc., San Francisco, CA (US)

(21) Appl. No.: 19/436,657

(22) Filed: Dec. 30, 2025

Related U.S. Application Data
(60) Provisional application No. 63/740,238, filed on Dec. 30, 2024.

(51) Int. Cl. A61B 5/06; A61B 5/00; A61B 90/00

(52) U.S. Cl. CPC A61B 5/06; A61B 5/7221; A61B 5/7405; A61B 5/742; A61B 5/7455; A61B 2090/065

(57) ABSTRACT

Scanning techniques to determine sensor placement are described. In an example, an indication is generated to place a scanning device at one or more measurement locations within a measurement region on an individual. A signal is obtained, by the scanning device, at each of the one or more measurement locations. The signal obtained at the one or more measurement locations is analyzed to determine a recommended location for placement of a monitoring device. The recommended location is output by the scanning device.

### RELATED APPLICATIONS

This application claims priority to U.S. Provisional Application No. 63/740,238, filed Dec. 30, 2024, and titled “Scanning Techniques to Determine Sensor Placement,” which is hereby incorporated by reference in its entirety.

### BACKGROUND

Photoplethysmography (PPG) sensors are widely used for non-invasive monitoring of various physiological parameters. For instance, PPG sensors measure changes in blood volume in microvascular tissue beds by detecting variations in light absorption. Using conventional techniques, PPG sensors are applied to peripheral sites with high perfusion and thin skin, such as fingers or earlobes. While such sites may be attractive for PPG measurements due to their high perfusion and accessibility, motion artifacts, pressure sensitivity, and ambient light interference at these sites can impact measurements. Additionally, such sites may be impractical or unsuitable for various applications, such as continuous monitoring during sleep or physical activity, creating a desire to explore alternative PPG sensor placement locations. However, an effectiveness of PPG measurements is highly dependent on sensor placement, as factors like local tissue perfusion, anatomical variations, and motion artifacts can significantly impact signal quality.

### BRIEF DESCRIPTION OF THE DRAWINGS

FIG. 1 is a block diagram of a non-limiting example of an environment that is operable to employ scanning techniques to determine sensor placement as described herein.

FIG. 2 depicts a non-limiting example of a monitoring device.

FIG. 3 illustrates a non-limiting example of a smartphone implementation of the scanning device.

FIG. 4 illustrates a non-limiting example of a user interface displaying a measurement region to be scanned using the scanning device.

FIG. 5 illustrates a non-limiting example of a sequence of user interfaces output during measurement location scanning by the scanning device.

FIG. 6 illustrates a non-limiting example of a sequence of user interfaces output by the scanning device during analysis of the scanning data.

FIG. 7 illustrates a non-limiting example of the scanning device receiving user input.

FIG. 8 illustrates a non-limiting example of a result of the scanning process using the scanning device.

FIG. 9 illustrates a flowchart of a method for determining a location for placement of a sensor.

### DETAILED DESCRIPTION

Conventional techniques for photoplethysmography (PPG) sensor placement involve application of sensors to peripheral sites with high perfusion and thin skin, such as fingers or earlobes. However, these locations are often unsuitable and/or impractical for various applications, such as continuous monitoring. While modalities that support alternative PPG sensor placement locations, e.g., the chest, are desirable, an effectiveness of PPG measurements can be highly dependent on sensor placement. Factors like local tissue perfusion, anatomical variations, and motion artifacts can significantly impact signal quality, making it challenging to determine suitable locations for PPG sensor placement. Moreover, the chest region presents unique challenges compared to traditional peripheral sites, as chest anatomy and physiology differ greatly from person to person due to various factors including age, gender, height, weight, perfusion, and body composition. Due to all these factors, it may be difficult to apply a single approach to sensor design and application that would be globally effective.

To address these limitations, techniques to implement a scanning device are described to determine an effective location for PPG sensor placement on a particular region, such as a person's chest. In an example, the techniques described herein generate an indication to place the scanning device at multiple locations on the chest region. The scanning device, which may be a dedicated device and/or a smartphone with a modified flashlight, obtains PPG signals at each location. These signals are then analyzed to determine a recommended location for PPG sensor placement that optimizes signal quality. As used herein, the term “optimizing” and its derivatives may refer to a process of improving or enhancing one or more characteristics of the PPG signal quality and/or PPG sensor placement based on measurable signal characteristics such as signal-to-noise ratio, pulse amplitude, perfusion index, and/or other quantifiable signal quality metrics. The optimization process may include comparing numerical values of the measurable signal characteristics across multiple measurement locations and selecting a location that demonstrates the highest measured values, lowest noise levels, and/or best performance according to established measurement criteria. By way of example, optimization may be performed using algorithmic analysis that obtains the PPG signals, processes the obtained PPG signals, and ranks locations based on calculated performance scores derived from the obtained PPG signals. A location may be recommended for PPG sensor placement, for instance, when it ranks highest of the multiple measurement locations. In one or more implementations, the scanning device is configured to build a map (e.g., a coarse map) of perfusion over an area of interest by prompting a user to place the scanning device in different spots within the area of interest. These spots may be arranged in various configurations (e.g., a grid) over the upper left chest using anatomical markers like the sternum and armpit as reference points.

The scanning process can be further refined by incorporating anatomical data about the individual to determine the initial scanning locations. For example, anatomical data such as the individual's height, weight, gender, BMI, location of anatomical markers, and so forth may be used to determine a configuration of scanning locations. Additionally, the scanning device may include a force sensor to measure an amount of force applied during scanning. This allows for feedback to be provided to the user, such as to indicate a recommended force to press the scanning device against the chest region. Such feedback can be visual, tactile, or auditory feedback to support a variety of implementations. For example, visual feedback can include written messages, icons, colors, and/or numerical indicators displayed on a screen to guide pressure application. Tactile feedback, by way of example, can include haptic, electrotactile, vibrotactile, and/or kinesthetic feedback that may provide physical sensations regarding pressure application. Examples of auditory feedback include spoken prompts, system sounds, notification tones, and/or other audio cues that indicate when appropriate pressure is applied. The force sensor may be integrated into a skin-facing surface of the scanning device and may provide real-time feedback to help standardize the measurement process and reduce variability due to inconsistent pressure application.

As mentioned above, analyzing the PPG signals may include measuring various parameters, such as a signal-to-noise ratio, a pulse amplitude, and/or a perfusion index. In at least one example, an analysis algorithm generates a perfusion map based on the PPG signals. As used herein, a “perfusion map” may refer to a representation that indicates blood flow characteristics across a measurement area to indicate variations in tissue perfusion levels at different locations in the measurement area. Further, the scanning device may generate prompts to place the scanning device at additional locations to further refine the analysis based on the PPG signals. Once the recommended location is determined, the scanning device is configured to mark this spot on the chest region, ensuring consistent placement for future measurements. Moreover, the PPG signal analysis may be customized for specific applications, as different applications of PPG technology may prioritize different features of the PPG signal. For instance, applications focused on heart rate variability may prioritize locations with clear pulsatile waveforms and strong pulse amplitude, while those measuring blood oxygen saturation may emphasize areas with good perfusion and high perfusion index values. Applications monitoring respiratory rate may benefit from measurement locations where the respiratory-induced intensity variations in the PPG signal are more pronounced. The analysis algorithm may be adapted based on the intended application to adjust, for example, the number, spacing, and/or arrangement of measurement locations to increase the quality of the relevant aspects of the PPG signal for each use case.

This approach offers several advantages over conventional systems, such as by providing a personalized, data-driven method for determining effective PPG sensor placement, accounting for individual anatomical differences. By systematically scanning multiple locations, the techniques described herein further increase a likelihood of finding a high-quality signal, improving the accuracy and reliability of PPG measurements, and conserving computational resources that may otherwise be allocated to resolve signal inconsistencies. Moreover, the systematic approach described herein may be used to identify a true maximum in signal quality rather than a local maximum. The incorporation of force feedback helps to standardize the measurement process, reducing variability due to inconsistent application pressure. Furthermore, the ability to use a smartphone as the scanning device makes this technique widely accessible and cost-effective.

In one or more implementations, the scanning device may be configured to obtain signals other than PPG signals, such as electrical signals (e.g., ECG signals and/or impedance measurements), depending on the use case and the type of sensor for which optimal placement is being determined. For example, when determining optimal placement for an ECG sensor, the scanning device may analyze characteristics of the electrical signals such as R-wave amplitude, signal-to-noise ratio, QRS complex clarity, baseline stability, electrode-skin impedance, and/or P-wave and T-wave visibility. The scanning device may generate a signal quality map based on the electrical signal characteristics obtained at the measurement locations, where the signal quality map indicates variations in electrical signal quality across the measurement region. The recommended location may be determined based on the signal quality map by identifying locations that exhibit enhanced electrical signal characteristics for the intended application of the monitoring device. When the scanning device is implemented as a smartphone, one or more additional sensors configured to capture such alternative or additional signals may be included in an accessory that communicates with the smartphone via wired or wireless communication protocols.

In one or more other implementations, the scanning device may include multiple different sensor types to obtain multiple different signal types at the measurement locations. For example, the scanning device may obtain both PPG signals and electrical signals (e.g., ECG signals) at each of the measurement locations. The analysis platform may generate a signal quality map for each signal type, such as a perfusion map based on the PPG signals and an electrical signal quality map based on the ECG signals. The analysis platform may determine the recommended location for placement of a multi-sensor monitoring device by balancing signal quality characteristics across the different signal types. For instance, the analysis platform may identify a location that provides acceptable signal quality for both PPG and ECG measurements, even if that location does not represent the highest signal quality for either signal type individually. The analysis platform may apply weighting factors to the different signal types based on the intended application of the monitoring device, such that signal types of greater importance to the application are weighted more heavily when determining the recommended location. In some implementations, the analysis platform may present multiple candidate locations to the user along with signal quality metrics for each signal type at each candidate location, allowing the user to select a location based on the relative importance of the different signal types for the intended use case. In this way, the techniques described herein may be adapted for determining the placement location for a variety of different sensor types.

In some aspects, the techniques described herein relate to a method for determining a location for placement of a monitoring device, including: generating an indication to place a scanning device at one or more measurement locations within a measurement region on an individual; obtaining, by the scanning device, a signal at each of the one or more measurement locations; analyzing the signal obtained at the one or more measurement locations to determine a recommended location for the placement of the monitoring device; and outputting the recommended location.

In some aspects, the techniques described herein relate to a method, further including: receiving anatomical data about the individual; and determining the one or more measurement locations based in part on the anatomical data.

In some aspects, the techniques described herein relate to a method, wherein the measurement region is at least a portion of a chest region of the individual.

In some aspects, the techniques described herein relate to a method, wherein obtaining, by the scanning device, the signal at each of the one or more measurement locations includes contacting the scanning device with the individual at each of the one or more measurement locations, and the method further includes: measuring, at the one or more measurement locations and via at least one force sensor of the scanning device, a force of the scanning device against the individual; generating force feedback for pressing the scanning device against the individual based on the measured force relative to a recommended force; and presenting the force feedback via the scanning device.

In some aspects, the techniques described herein relate to a method, wherein the force feedback includes one or more of visual feedback, haptic feedback, or auditory feedback.

In some aspects, the techniques described herein relate to a method, further including: determining a quality of the signal obtained at the one or more measurement locations; and outputting a prompt to place the scanning device at one or more additional measurement locations within the measurement region based on the quality of the signal obtained at the one or more measurement locations.

In some aspects, the techniques described herein relate to a method, wherein the quality includes at least one of a signal-to-noise ratio, a pulse amplitude, a perfusion index, a consistency at rest, or a baseline wander, and wherein outputting the prompt to place the scanning device at the one or more additional measurement locations based on the quality of the signal obtained at the one or more measurement locations includes: generating a signal quality map by mapping the quality of the signal obtained at the one or more measurement locations to the measurement region of the individual; estimating a position within the measurement region that has a highest quality of the signal based on the signal quality map; and outputting the prompt to place the scanning device at the one or more additional measurement locations within the measurement region based on the estimated position.

In some aspects, the techniques described herein relate to a method, wherein the scanning device is configured to mark the individual at the recommended location.

In some aspects, the techniques described herein relate to a method, wherein the signal is a photoplethysmography (PPG) signal, and the scanning device includes a smartphone with a light source configured to emit light toward skin of the individual and a photodetector configured to measure reflected light from the skin to obtain the PPG signal.

In some aspects, the techniques described herein relate to a system for determining a placement location of a monitoring device, including: a scanning device having at least one sensor configured to obtain at least one physiological signal from an individual; at least one processor configured to: generate an indication to place the scanning device at one or more measurement locations within a measurement region on the individual; receive the at least one physiological signal obtained by the scanning device at the one or more measurement locations; and generate a recommended location for the placement location of the monitoring device within the measurement region based on the at least one physiological signal obtained at the one or more measurement locations; and a display configured to present the recommended location.

In some aspects, the techniques described herein relate to a system, wherein the at least one sensor includes a photoplethysmography (PPG) sensor configured to obtain a PPG signal from the individual, the PPG sensor including: a light source configured to emit light toward skin of the individual; and a photodetector configured to detect reflected light from the skin.

In some aspects, the techniques described herein relate to a system, wherein the at least one sensor includes an electrode configured to obtain an electrical signal from the individual.

In some aspects, the techniques described herein relate to a system, wherein the at least one sensor includes both of a PPG sensor configured to obtain a PPG signal from the individual and an electrode configured to obtain an electrical signal from the individual.

In some aspects, the techniques described herein relate to a system, wherein the at least one processor is further configured to: determine a signal quality of the at least one physiological signal obtained at the one or more measurement locations; generate at least one signal quality map based on the signal quality of the at least one physiological signal obtained at the one or more measurement locations; and generate the recommended location for the placement location of the monitoring device based on the at least one signal quality map.

In some aspects, the techniques described herein relate to a system, wherein the signal quality includes at least one of a signal-to-noise ratio, an amplitude, a perfusion index, a consistency at rest, a baseline wander, a QRS complex clarity, a baseline stability, an electrode-skin impedance, a P-wave visibility, or a T-wave visibility.

In some aspects, the techniques described herein relate to a system, wherein the scanning device further includes: at least one force sensor positioned on a skin-facing surface of the scanning device, the at least one force sensor configured to measure an amount of force applied to skin of the individual by the scanning device; and at least one physical alignment on the skin-facing surface and configured to mark the individual at the recommended location when contacted with the skin of the individual.

In some aspects, the techniques described herein relate to a method for determining a location for placement of a monitoring device having at least one sensor, including: generating, via a scanning device, an indication to place the scanning device at one or more measurement locations within a measurement region on an individual; obtaining, via the scanning device, at least one signal at each of the one or more measurement locations; generating, by an analysis platform, a signal quality map of the measurement region based on the at least one signal at each one of the one or more measurement locations; determining, by the analysis platform, a recommended location for the placement of the monitoring device based on the signal quality map; and indicating the recommended location via the scanning device.

In some aspects, the techniques described herein relate to a method, further including: receiving anatomical data about the individual; and determining the one or more measurement locations based at least in part on the anatomical data.

In some aspects, the techniques described herein relate to a method, wherein the measurement region is within a chest region of the individual.

In some aspects, the techniques described herein relate to a method, wherein the at least one signal includes both of a photoplethysmography (PPG) signal and an electrical signal, and determining, by the analysis platform, the recommended location for the placement of the monitoring device based on the signal quality map includes: generating a perfusion map based on the PPG signal; generating an electrical signal quality map based on the electrical signal; and applying weighting factors to the perfusion map and the electrical signal quality map based on an intended application of the monitoring device to determine the recommended location.

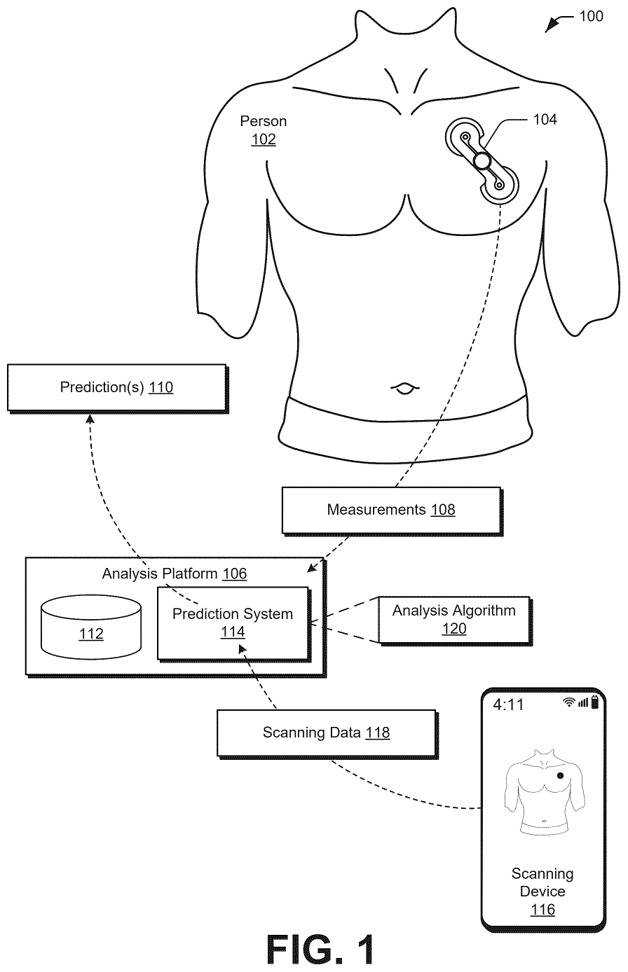

FIG. 1 is a block diagram of a non-limiting example 100 of an environment that is operable to employ scanning techniques to determine sensor placement as described herein. The illustrated example 100 includes a monitored subject, e.g., a person 102, who is depicted wearing a monitoring device 104. The illustrated environment also includes an analysis platform 106. The analysis platform 106 may be connected to the monitoring device 104 via one or more wireless connections directly or via one or more wired and/or wireless connections and one or more intermediate devices, such as a computing device associated with the person 102, network routing devices and equipment, server devices, and/or the Internet, to name just a few.

The monitoring device 104 may be utilized to monitor one or more aspects of the person 102. By way of example, the monitoring device 104 may be utilized to monitor one or more of electrocardiography (ECG), electroencephalography (EEG), electromyography (EMG), respiratory inductance plethysmography (RIP), photoplethysmography (PPG), accelerometry, or the like as measurements 108. For instance, the monitoring device 104 may include a PPG sensor for non-invasive monitoring of various physiological parameters. By way of example, the PPG sensor may be utilized to monitor one or more of heart rate, heart rate variability, blood oxygen saturation, respiration, blood volume, and blood perfusion. The PPG sensor may comprise one or more light sources, such as light-emitting diodes (LEDs) and/or laser diodes, and one or more photodetectors. The one or more light sources may emit light at one or more wavelengths to monitor the various physiological parameters. By way of example, the one or more light sources may emit light in the red to infrared spectrum, which penetrates the skin and underlying tissues more efficiently than light having shorter wavelengths (e.g., light within the ultraviolet to orange regions of the spectrum). In at least one variation, however, the one or more light sources of the PPG sensor emit light of a shorter wavelength (e.g., green light) in addition to or as an alternative to the longer wavelength light. In one or more implementations, the PPG sensor is configured to emit and detect multiple different wavelengths of light to capture different physiological parameters.

As the heart pumps blood through the body, the volume of blood in the microvascular bed of the tissue fluctuates. The PPG sensor detects these volume changes by measuring the amount of light reflected or transmitted by the blood vessels. The photodetector captures the reflected or transmitted light, which varies with each heartbeat, allowing the device to measure parameters such as heart rate, blood oxygen saturation, and other pulse wave characteristics.

The monitoring device 104 may process raw PPG signals on-board or transmit the data to the analysis platform 106 for further analysis. In some cases, the PPG sensor in the monitoring device 104 may be designed for continuous monitoring, allowing for long-term tracking of various health metrics. The device may also incorporate algorithms to filter out motion artifacts and other noise, improving the accuracy of measurements.

In some aspects, the monitoring device 104 may combine PPG sensing with other modalities, such as ECG or accelerometry, to provide a more comprehensive picture of the physiological state of the person 102. This multi-modal approach may enhance the ability of the monitoring device 104 to detect and monitor various health conditions, including sleep disorders, arrhythmias, or changes in cardiovascular function.

In some scenarios, for instance, the monitoring device 104 may be provided to record electrical activity of the person 102's heart over an observation period, e.g., lasting some number of seconds or minutes, lasting multiple days, and so on. By way of example, the person 102 may have a magnitude of his or her heart's electrical potential monitored over time to produce one or more electrocardiograms, which may be used to predict any of a variety of events. Alternatively, or in addition, the monitoring device 104 may be used to output the measurements 108 (e.g., a time sequence of measurements such as a time sequence of electric potential measurements), which may indicate an observation or be used to generate a prediction of one or more events.

As used herein, the term “continuous” used in connection with the measurements 108 may refer to an ability of a device to produce measurements substantially continuously, such that the device may be configured to produce the measurements 108 as outputs at intervals of time (e.g., per hour, per 30 minute interval, per 5 minute interval, per 30 second interval, per second, per half second, and so forth), responsive to an event (e.g., an electrical signal reaching an inflection point such as a peak or a valley), and so forth. The functionality of the monitoring device 104 to produce the measurements 108 and/or to record any of a variety of signals may vary without departing from the spirit or scope of the described techniques.

In connection with the monitoring device 104, instructions may be provided to the person 102 that instruct the person 102 how to operate the monitoring device 104 and/or how to behave (e.g., sleep, perform activity) while wearing monitoring device 104. In one or more implementations, the instructions may be provided as part of a kit, e.g., written instructions. Alternatively, or additionally, the analysis platform 106 may cause the instructions to be communicated to and output (e.g., for display and/or audio output) via a computing device associated with the person 102. In one or more implementations, the analysis platform 106 may wait to provide these instructions for output after a predetermined amount of time of an observation period has lapsed (e.g., two days) while wearing the monitoring device 104 and/or based on patterns in the aspects of the person 102 being measured.

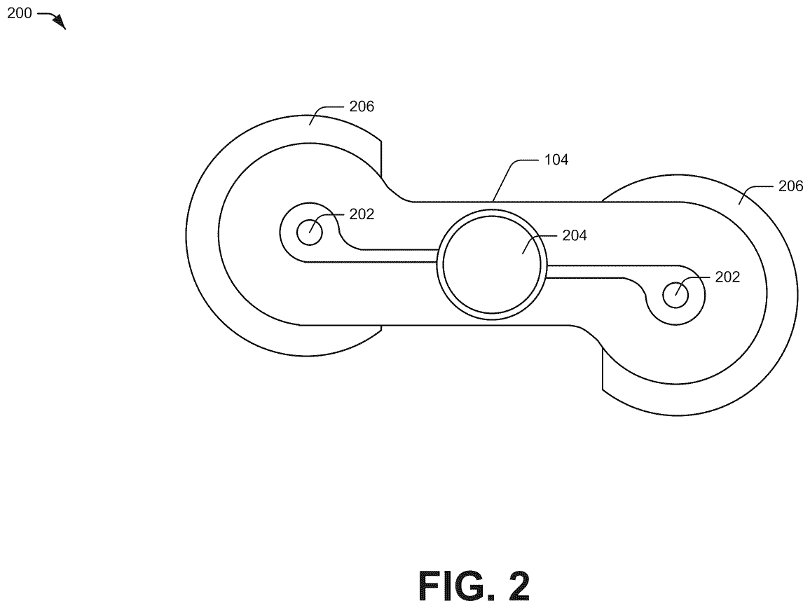

The monitoring device 104 may be configured in a variety of ways to monitor one or more aspects of the person 102. Moreover, the form factor depicted in FIGS. 1 and 2 is just one example form factor, and the form factor of the monitoring device 104 may differ in variations. It is to be appreciated that the monitoring device 104 may be configured with one or more sensors, examples of which include one or more of: a plurality of electrodes (e.g., that can be placed on the skin of the person), an accelerometer, and a PPG sensor (e.g., to measure and record oxygen saturation (SpO2) and/or produce a photoplethysmogram of the person 102), to name just a few. Certainly, the monitoring device 104 may be configured with any of a variety of types of sensors without departing from the described techniques.

Although the monitoring device 104 may be configured in a similar manner as monitoring devices used for clinically monitoring patients, in one or more implementations, the monitoring device 104 may be configured differently than the devices used for monitoring and/or diagnosing patients clinically. By way of example, and not limitation, the monitoring device 104 may be configured as a ring, a watch, a patch, and/or a strap, to name just a few form factors. Alternatively, or additionally, the monitoring device 104 may have a similar form factor as for clinical settings, but may have different functionality, such as functionality that prevents a wearer from viewing the measurements.

In one or more implementations, the monitoring device 104 may be configured to offload measurements and/or other data from the monitoring device during the course of the observation period. By way of example, the monitoring device 104 may offload the measurements by transmitting them via a wired or wireless connection to an external computing device, e.g., at predetermined time intervals and/or responsive to establishing or reestablishing a connection with the computing device. In one or more implementations, the measurements 108 and/or other data from the monitoring device 104 may be compressed by the monitoring device 104 for wireless transmission, e.g., using one or more of a variety of data compression techniques. Compression of the sensor data in this way can reduce battery usage of the monitoring device 104 during the observation period and facilitate wear during assessments of sleep apnea.

To the extent that the monitoring device 104 may be configured to store the measurements 108 for an entirety of an observation period, in one or more implementations, the monitoring device 104 may be configured without wireless transmission means, e.g., without any antennae to transmit the measurements 108 wirelessly and without hardware or firmware to generate packets for such wireless transmission. Instead, the monitoring device 104 may be configured with hardware to communicate the measurements 108 via a physical, wired coupling. In such scenarios, the monitoring device 104 may be “plugged in” to extract the measurements 108 from the device's storage.

Accordingly, the monitoring device 104 may be configured with one or more ports to enable wired transmission of the measurements to an external computing device. Examples of such physical couplings may include micro universal serial bus (USB) connections, mini-USB connections, and USB-C connections, to name just a few. Although the monitoring device 104 may be configured for extraction of the measurements 108 via wired connections as discussed just above, in different scenarios, the monitoring device 104 may alternately or additionally be configured to offload the measurements 108 over one or more wireless connections.

Once the monitoring device 104 produces the measurements 108, the measurements are provided to the analysis platform 106. As noted above, the measurements 108 may be communicated to the analysis platform 106 over wired and/or wireless connection(s).

In scenarios where the analysis platform 106 is implemented partially or entirely on the monitoring device 104, for instance, the measurements 108 may be transferred over a bus from the device's local storage to a processing system of the device. In scenarios where the monitoring device 104 is configured to generate one or more predictions 110 by processing the measurements 108, the monitoring device 104 may also be configured to provide the generated one or more predictions 110 as output, e.g., by communicating the one or more predictions 110 to an external computing device. In other scenarios, the measurements 108 may be processed by an external computing device configured to generate the one or more predictions 110. For example, the measurements 108 (and/or other measurements such as accelerometer data and ECG data) may be processed by a smartphone associated with the user, a smartphone or other dedicated device associated with the monitoring device 104, and/or one or more server computers at a data center or other location that can be utilized by an entity associated with the monitoring device 104, to name just a few.

In one or more implementations, the monitoring device 104 is configured to transmit the measurements 108 to an external device over a wired connection with the external device, e.g., via USB-C or some other physical, communicative coupling. Here, a connector may be plugged into the monitoring device 104 or the monitoring device 104 may be inserted into an apparatus having a receptacle that interfaces with corresponding contacts of the device. The measurements 108 may then be obtained from storage of the monitoring device 104 via this wired connection, e.g., transferred over the wired connection to the external device. Such a connection may be used in scenarios where the monitoring device 104 is mailed by the person 102 after the observation period, such as to a health care provider, telemedicine service, provider of the monitoring device 104, or medical testing laboratory.

Alternatively, or additionally, the monitoring device 104 may provide the measurements 108 to the analysis platform 106 by communicating the measurements 108 over one or more wireless connections. For example, the monitoring device 104 may wirelessly communicate the measurements 108 to external computing devices, such as a mobile phone, tablet device, laptop, smart watch, other wearable health tracker, and so on. Accordingly, the monitoring device 104 may be configured to communicate with external devices using one or more wireless communication protocols or techniques. By way of example, the monitoring device 104 may communicate with external devices using one or more of Bluetooth® (e.g., Bluetooth® Low Energy links), near-field communication (NFC), Long Term Evolution (LTE™) standards such as 5G, and so forth. The monitoring device 104 may be configured with corresponding antennae and other wireless transmission means in scenarios where the measurements 108 are communicated to an external device for processing. In those scenarios, the measurements 108 may be communicated to the analysis platform 106 in various manners, such as at predetermined time intervals (e.g., every day, every hour, or every five minutes), responsive to occurrence of some event (e.g., filling a storage buffer of the monitoring device 104), or responsive to an end of an observation period, to name just a few.

Thus, regardless of where the analysis platform 106 is implemented (e.g., at the monitoring device 104, at a smartphone associated with the person 102, or at a server device), the analysis platform 106 obtains the measurements 108 produced by the monitoring device 104. In one or more implementations, the analysis platform 106 also obtains other measurements produced by the monitoring device 104 and/or any other devices used during the observation period, e.g., a smart watch, chest strap, etc. As noted above, examples of such additional measurements include but are not limited to accelerometer data and/or ECG data.

In one or more implementations, the analysis platform 106 may be implemented in whole or in part at the monitoring device 104. Alternatively or in addition, the analysis platform 106 may be implemented in whole or in part using one or more computing devices external to the monitoring device 104, such as one or more computing devices associated with the person 102 (e.g., a mobile phone, tablet device, laptop, desktop, or smart watch) or one or more computing devices associated with a service provider (e.g., a health care provider, a telemedicine service, a service corresponding to the provider of the monitoring device 104, a medical testing laboratory service, and so forth). In the latter scenario, the analysis platform 106 may be implemented at least in part on one or more server devices.

In the illustrated example 100, the analysis platform 106 includes a storage device 112 and a prediction system 114. In accordance with the described techniques, the storage device 112 is configured to maintain the measurements 108 and/or other measurements or information processed by the prediction system 114 to generate the one or more predictions 110. The storage device 112 may represent one or more databases and/or other types of storage capable of storing the measurements 108 and/or other types of measurements. The storage device 112 may also store a variety of other data, such as personal information, demographic information describing the person 102, information about a health care provider, information about an insurance provider, payment information, prescription information, determined health indicators, account information (e.g., username and password), and so forth. The storage device 112 may also maintain data of other users of a user population.

In the illustrated example 100, the prediction system 114 represents functionality to process the measurements 108 to generate the one or more predictions 110. Alternatively, or in addition, the prediction system 114 may output one or more time sequences indicating an observation or prediction of one or more events over time. It is also to be appreciated that, in variations, the prediction system 114 may output different combinations of multiple predictions.

In at least one implementation, the prediction system 114 uses machine learning to generate at least a portion of the one or more predictions 110. By way of example and not limitation, the prediction system 114 may include one or more neural networks trained based on the historical measurements and the historical outcome data of a user population. The prediction system 114 may include one or multiple machine learning models (e.g., an ensemble of models). Alternatively, or additionally, the prediction system 114 may include logic (a machine learning model and/or other types of logic) to pre-process the measurements 108, such as to extract various cardiovascular and/or other features from the sequences of measurements. In the illustrated example 100, for instance, the one or more predictions 110 correspond to the output of the prediction system 114.

The illustrated example 100 further includes a scanning device 116. The scanning device 116 is configured to implement the techniques described herein to determine an effective location for placement of a photoplethysmography (PPG) sensor, e.g., the monitoring device 104. In some cases, the scanning device 116 may be configured for PPG measurement, such as including one or more sensors, properties, and/or functionality as described above with respect to the monitoring device 104. The scanning device 116 may communicate with the analysis platform 106 to transmit scanning data 118 to the prediction system 114. The scanning data 118 may include PPG measurements obtained by the scanning device 116 at one or more measurement locations on the person 102. A process referred to herein as “performing a scan” may include obtaining the scanning data 118 at a measurement location. In various examples, the prediction system 114 is configured to generate the one or more predictions 110 as described above based on the scanning data 118.

In some implementations, the scanning device 116 includes a force sensor. The force sensor of the scanning device 116 may measure a force of the scanning device 116 against a region of the person 102, e.g., a chest region. Based on the force measurement, the scanning device 116 may present feedback that indicates a recommended force to use when pressing the scanning device 116 against the chest region. The feedback may include one or more of visual feedback, tactile feedback, or auditory feedback.

In some cases, the scanning device 116 may be integrated with a smartphone and a smartphone app (e.g., an application executing on the smartphone) for user interface and software functionality. For example, the scanning device 116 may utilize a smartphone's flashlight and camera to perform PPG measurements. The smartphone app may provide instructions to guide a user through the scanning process, display real-time PPG signal quality, and present the recommended sensor placement location. Integrating the scanning device 116 with a smartphone may leverage existing hardware components, making the technology widely accessible without relying on specialized equipment. The smartphone app, for instance, may include features such as step-by-step guidance through the PPG measurement process, visual indicators for device positioning, and/or real-time signal quality feedback during measurements. In one or more implementations, the smartphone app may store measurement results for future reference. Alternatively, or in addition, the smartphone app may provide customization options based on characteristics of the person 102 such as age, gender, height, weight, and/or BMI. Additionally, the smartphone app may include educational content about proper measurement techniques, troubleshooting guides for common issues, and integration with other health monitoring applications.

The scanning device 116 may work in conjunction with the monitoring device 104 and the analysis platform 106 to determine one or more effective PPG sensor placement locations. For instance, the scanning device 116 may obtain PPG signals at multiple locations on the chest region of the person 102, e.g., within a targeted measurement region. The PPG signals may be transmitted as the scanning data 118 to the analysis platform 106. The prediction system 114 of the analysis platform 106 may analyze the scanning data 118 (optionally, along with the measurements 108 from the monitoring device 104) to determine a recommended location for PPG sensor placement. The analysis platform 106 may generate the one or more predictions 110, which may indicate the sensor placement location and may be communicated back to the scanning device 116 for presentation to the user. In at least one implementation, the recommended location for PPG sensor placement is a recommended location for placing the monitoring device 104, such as when the monitoring device 104 has not yet been placed on the person 102. If the monitoring device 104 has already been placed on the person 102, the measurements 108 may be analyzed in conjunction with new or prior scanning data 118 to determine the recommended location for placement. If the monitoring device 104 is repositionable, this process may be repeated to confirm the recommended location for placing the monitoring device 104.

In one or more implementations, the prediction system 114 includes an analysis algorithm 120 configured to process the scanning data 118 to determine the recommended location for placement of the monitoring device 104, e.g., based on a signal quality of the PPG signal obtained in the scanning data 118. The analysis algorithm 120, for example, may analyze various characteristics of scanning data 118, such as a signal-to-noise ratio, a pulse amplitude, a perfusion index, a waveform morphology, a consistency at rest, a baseline wander, a pulse peak strength, and/or a signal stability. In some implementations, the analysis algorithm 120 may be configured and/or adjusted based on an intended application of the monitoring device 104. For instance, applications focused on heart rate variability may cause the analysis algorithm 120 to prioritize (e.g., more highly rank) locations with clear pulsatile waveforms and strong pulse amplitude when assessing the signal quality, while applications measuring blood oxygen saturation may cause the analysis algorithm 120 to prioritize areas with high perfusion index values. As another example, the analysis algorithm 120 may prioritize measurement locations where respiratory-induced intensity variations in the PPG signal are more pronounced for applications monitoring respiration rate.

In one or more implementations, the analysis algorithm 120 may generate a perfusion map by converting the scanning data 118 into a representation that indicates blood flow characteristics across the measurement region and determine the recommended location therefrom, e.g., at a location of highest perfusion. Alternatively, or in addition, the analysis algorithm 120 may compare signal quality metrics across the measurement locations and rank the locations based on one or more selection criteria to identify the recommended location for placement of the monitoring device 104. When the monitoring device 104 includes multiple sensor types, the analysis algorithm 120 may balance signal quality characteristics across the different signal types to determine the recommended location. For example, the analysis algorithm 120 may apply weighting factors to different signal types based on the intended application and identify a location that provides acceptable signal quality across the multiple sensor types. The analysis algorithm 120 may further determine one or more additional measurement locations for obtaining the scanning data 118 within the measurement region based on the perfusion map and/or the ranked locations, which may prompt the scanning device 116 to guide a user to perform additional scans.

In some examples, one or more of the monitoring device 104, the scanning device 116, and/or the analysis platform 106 may be integrated into a consolidated device that performs the functionality of each of the analysis platform 106, the monitoring device 104, and/or the scanning device 116. For example, the consolidated device incorporates sensors and capabilities of the monitoring device 104 as well as scanning and analysis features of the scanning device 116.

The consolidated device may include the sensors of the monitoring device 104, such as electrodes for ECG measurements, EMG measurements, accelerometers, PPG sensors, and sweat sensors. It may also incorporate the PPG measurement capabilities and force sensors of the scanning device 116. Alternatively, or in addition, the scanning device 116 may include sensors for collecting electrical information, such as impedance measurements. This integration allows for comprehensive physiological monitoring and determination of optimal sensor placement within a single device. In such configurations, the consolidated device may perform continuous monitoring functions like the monitoring device 104, while also enabling the scanning and placement optimization features of the scanning device 116. The device may switch between monitoring and scanning modes and/or perform both functions simultaneously.

The consolidated device may utilize a single processing unit to handle both the monitoring and scanning functionalities. It may include enhanced wireless communication capabilities to transmit both the measurements 108 and the scanning data 118 to the analysis platform 106. The device may also incorporate a display or interface to provide user feedback for both monitoring status and sensor placement guidance.

By combining the functionality of both the monitoring device 104 and the scanning device 116, the consolidated device may offer a streamlined user experience by reducing the number of devices used and simplifying the overall monitoring and optimization process. This integration may also allow for more sophisticated analysis by leveraging data from both monitoring and scanning functions in real-time.

FIG. 2 depicts a non-limiting example 200 of a monitoring device. The illustrated example 200 depicts the monitoring device 104.

In accordance with the described techniques, the monitoring device 104 includes one or more sensors 202, examples of which include but are not limited to one or more pairs of electrodes, an accelerometer, a PPG sensor, and sweat sensors, to name just a few. As described above in more detail, in various examples, the monitoring device 104 is configured for pulse oximetry using the PPG signal(s) obtained by the PPG sensor. By way of example, the pulse oximetry may use PPG signals obtained at one or more wavelengths (e.g., typically red and infrared wavelengths) to measure blood oxygen saturation (e.g., SpO2) by analyzing the differential absorption characteristics of oxygenated and deoxygenated hemoglobin in the blood.

The monitoring device 104 may also include a transmitter 204. In this example 200, the monitoring device 104 further includes one or more adhesive portions 206. In operation, the monitoring device 104 is configured to be applied to the skin via the one or more adhesive portions 206, such that, for example, the one or more sensors 202 are positioned to detect and record the electrical activity of the person 102's heart, e.g., to produce an electrocardiogram (ECG and/or EKG). In at least one implementation, the monitoring device 104 may be removed by peeling the one or more adhesive portions 206 off of the skin.

It is to be appreciated that the monitoring device 104 and its various components are simply one form factor, and the monitoring device 104 and its components may have different form factors without departing from the spirit or scope of the described techniques.

In one or more implementations, the monitoring device 104 may include a processor and/or memory (not shown). The monitoring device 104, by leveraging the processor, may generate the measurements 108 based on the communications with the one or more sensors 202 that are indicative of some aspect of the person 102, such as the person 102's heart's electrical activity. In one or more implementations, the processor further generates one or more communicable packages of data that include one or more of the measurements 108 and/or other measurements, such as the scanning data 118. Alternatively, or additionally, the processor produces and/or causes storage of other data, which may be used for monitoring various physiological states of the person 102.

In implementations where the monitoring device 104 is configured for wireless transmission, the transmitter 204 may transmit the measurements wirelessly as a stream of data to a computing device. In one or more implementations, for instance, the monitoring device 104 is configured to transfer (e.g., transmit and/or receive) information (e.g., PPG measurements) via a Bluetooth® Low Energy (BLE) connection. Alternately or additionally, the monitoring device 104 may buffer the measurements (e.g., in memory) and cause the transmitter 204 to transmit the buffered measurements later at various intervals, e.g., time intervals (every second, every thirty seconds, every minute, every five minutes, every hour, and so on), storage intervals (when the buffered measurements reach a threshold amount of data), and so forth.

### Scanning Techniques to Determine Sensor Placement

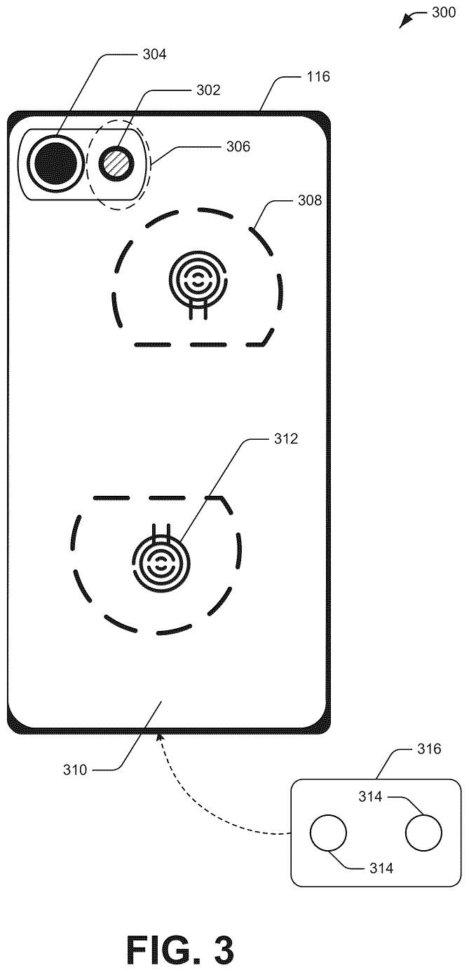

FIG. 3 illustrates a non-limiting example 300 of a smartphone implementation of the scanning device 116. The scanning device 116 includes a light source 302 for transmitting light into the person 102 and a photodetector 304 that captures light reflected from the person 102. The light source 302, for instance, may be a light-emitting diode, a laser diode, or another type of light source configured to emit light of a desired wavelength (or wavelengths) for obtaining the scanning data 118. The photodetector 304 may include an image sensor, such as a charge-coupled device or a complementary metal-oxide-semiconductor sensor. In at least one variation, the photodetector 304 is a photodiode or a phototransistor.

It is to be appreciated that in at least one variation, the scanning device 116 includes more than one light source 302, more than one photodetector 304, and/or is otherwise adapted to emit and detect light at multiple locations simultaneously. By way of example, the scanning device 116 may optionally include an adapter 306 positioned over the light source 302 to direct or disperse light emitted by the light source 302 into a grid pattern, where the grid pattern enables simultaneous assessment of PPG signal quality at multiple measurement locations. The adapter 306 may include one or more optical elements such as lenses, prisms, and/or diffraction gratings configured to split and/or redirect the light from the light source 302 to illuminate multiple discrete areas. The photodetector 304 may be configured to capture reflected light from each of the illuminated areas, allowing the scanning device 116 to obtain the scanning data 118 from multiple measurement locations concurrently. This simultaneous measurement approach may reduce the number of iterative or down-selecting measurements used before determining a recommended location, thereby streamlining the scanning process.

The scanning device 116 of the example 300 is configured to mark a recommended location (or another scanned location) on the person 102. It will be appreciated that any form of physical marking is contemplated. In an example, the mark is temporary and configured to disappear after an amount of time passes that is sufficient for placing the PPG sensor at the recommended location. To do so, the scanning device 116 may include a physical alignment aid 308 on an external, e.g., skin-facing surface 310 of the scanning device 116 to mark the skin of the person 102. The physical alignment aid 308 may include a raised edge or another type of mark making feature. When pressed against the skin, for example, the raised edge may create a temporary indent, serving as a physical marker for the sensor placement. In the example 300, the raised edge is shaped to correspond with the edges of the one or more adhesive portions 206 of the monitoring device 104. Alternatively, the physical alignment aid 308 can correspond with the center of the monitoring device 104 or another portion of the monitoring device 104. The physical alignment aid 308 may take any shape and be located anywhere along skin-facing surface 310. In at least one example, ink can be applied to at least a portion of the physical alignment aid 308, whereby the ink is configured to transfer to the skin of the person 102 when contacted to the person 102.

In the example 300, the physical alignment aid 308 includes a series of equally spaced segments having the same length. Alternatively, the segments may be unequally spaced and have differing lengths. In an alternative example, the physical alignment aid 308 may be a continuous, unbreaking segment.

The scanning device 116 of the example 300 further includes one or more force sensors 312 configured to measure a pressure applied during scanning. As a non-limiting example, the scanning device 116 includes two force sensors 312. It will be appreciated, however, that the scanning device 116 can have any number of force sensors 312. The one or more force sensors 312 of the example 300 may be positioned anywhere along the skin-facing surface 310. As a non-limiting example, the scanning device 116 may include one force sensor 312 located in the center of the skin-facing surface 310.

The one or more force sensors 312 are configured to measure an amount of force applied while the skin-facing surface 310 is contacted to the person 102, such as while acquiring the scanning data 118 and/or before acquiring the scanning data 118. The force measurements obtained by the one or more force sensors 312 may be used by the analysis platform 106 and/or the scanning device 116 to output feedback regarding the amount of force applied relative to a desired (e.g., recommended) amount of force for obtaining the scanning data 118. By way of example, the analysis algorithm 120 may compare the force measurements obtained by the one or more force sensors 312 to the desired amount of force and generate the feedback (e.g., real-time feedback) responsive thereto. The feedback may be output via the scanning device 116, for instance, and may indicate either adjusting or maintaining the amount of force applied via the scanning device 116. For example, the feedback may indicate increasing the applied force in response to the applied force being less than the desired amount of force, indicate maintaining the applied force in response to the applied force being equal to the desired amount of force, or indicate decreasing the applied force in response to the applied force being greater than the desired amount of force. The feedback may be visual, tactile, and/or auditory in nature. By way of non-limiting example, the scanning device 116 may display a visual textual message indicating to reduce, increase, or maintain the applied force based on the measurements obtained by the one or more force sensors 312.

By providing a physical marking method via the physical alignment aid 308, the scanning device 116 may enable accurate and consistent placement of the monitoring device 104 and/or the one or more sensors 202 at the determined location, which may help improve the quality and reliability of subsequent PPG measurements taken by the monitoring device 104. It is to be appreciated that the physical marking method may be applied one or more times to aid placement. For example, the physical mark created by the physical alignment aid 308 may be applied at the recommended location determined by the analysis algorithm 120. Alternatively, the physical mark created by the physical alignment aid 308 may be applied each time the scanning device 116 makes a scan during the scanning analysis, which may help ensure even and comprehensive coverage over the scanning area.

In one or more implementations, the scanning device 116 includes one or more additional sensors 314 configured to capture additional or alternative signals, such as electrical signals (e.g., ECG and/or impedance measurements). In instances where the scanning device 116 is implemented as a smartphone, such as illustrated in the example 300, the one or more additional sensors 314 may be included in an accessory 316 that is in electronic communication with the smartphone. The accessory 316 may communicate with the smartphone via its charging port, data transmission over Bluetooth®, and/or other wireless or wired communication protocols. The selection of signals captured by the scanning device 116 may be tailored to the specific use case and/or the type of sensor for which location placement is being determined. For example, when determining placement for an ECG sensor, the scanning device 116 may prioritize electrical signal measurements. In some instances, the scanning device 116 may be implemented as a custom device specifically designed to capture the signals most relevant for determining optimal placement for a given sensor type. Accordingly, it is to be appreciated that the smartphone implementation is provided by way of illustration and not limitation.

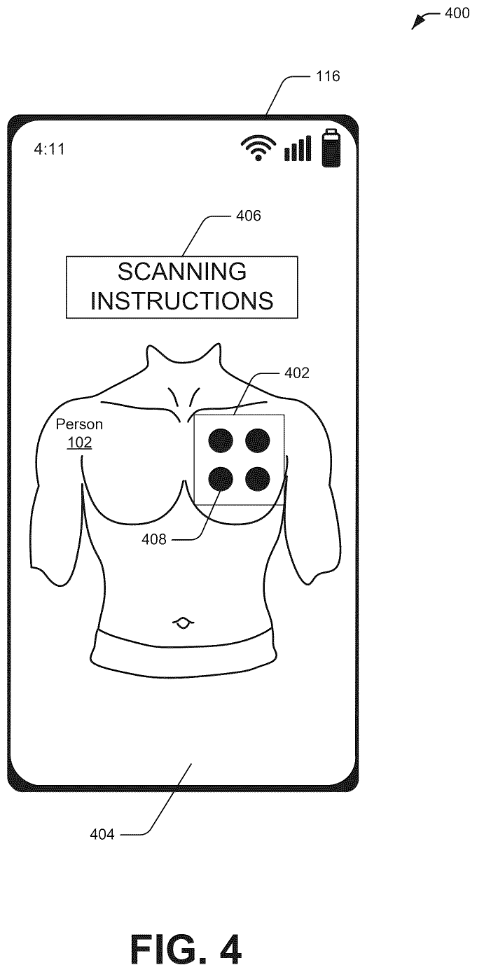

FIG. 4 illustrates a non-limiting example 400 of a user interface displaying a measurement region 402 to be scanned using the scanning device 116. In the example 400, the scanning device 116 is implemented as a smartphone, and a display screen 404 of the scanning device 116 displays the measurement region 402 along with scanning instructions 406. The scanning device 116 may be configured to measure PPG signals such as by using a flashlight and/or camera of the smartphone (e.g., the light source 302 and the photodetector 304, respectively). Alternatively, or in addition, the scanning device 116 may be configured to measure electrical signals or another type of signal obtained by the one or more additional sensors 314.

In the example 400, the scanning device 116 displays the scanning instructions 406 at the top portion of the display screen 404, above the measurement region 402. In variations, however, the scanning instructions 406 may be positioned adjacent to the measurement region 402 or below the measurement region 402. The scanning instructions 406 provide guidance to a user regarding where to position the scanning device 116 on the person 102. The user may be the person 102 or another person who is helping the person 102, for example. The scanning device 116 may be configured to read the scanning instructions 406 aloud with spoken prompts and/or may produce other forms of auditory and/or non-auditory feedback. Alternatively, or in addition, the scanning instructions 406 may be configured as a pop-up for the user to click through. By way of example, the scanning instructions 406 may overlay the measurement region 402 to prompt the user to acknowledge the scanning instructions 406 before the measurement region 402 is displayed. As a further example, the scanning instructions 406 may be configured to disappear after the passage of a predetermined amount of time, e.g., 30 seconds, a minute, or another time duration.

In the example 400, the measurement region 402 is shown overlaid on the upper left chest region of the person 102. The measurement region 402 indicates one or more measurement locations 408 in a region of interest (e.g., the chest region) to place the scanning device 116. In this example, the measurement region 402 includes four measurement locations 408 arranged in a 2×2 grid configuration over an upper left quadrant of the chest region of the person 102. This is by way of example and not limitation, and a variety of location configurations and positions are considered. For example, the measurement region 402 can include nine measurement locations 408 arranged in a 3×3 configuration, sixteen measurement locations 408 arranged in a 4×4 configuration, twenty-five measurement locations 408 arranged in a 5×5 configuration, and so on.

The measurement locations 408 may be arranged in a grid having at least one row and at least one column, where the number of rows and columns can be the same or differ from one another. In at least one variation, the measurement locations 408 may be arranged in another type of ordered pattern or in an irregular pattern within the measurement region 402. As a further example, the measurement region 402 may be irregularly shaped, and the measurement locations 408 are not restricted to a grid configuration. By way of example and not limitation, the measurement region 402 may be shaped to flexibly indicate multiple measurement locations 408 in a hierarchical manner based on anatomical data like height, weight, gender, BMI, locations of anatomical markers, and so forth. In one or more implementations, the configuration of the measurement region 402 may be dynamically adjusted based on the size and shape of the region of interest, e.g., with larger regions of interest having more measurement locations to adequately map signal quality and/or perfusion characteristics of the larger region of interest. The spacing between the measurement locations 408 may also be adjusted based on anatomical considerations, with closer spacing in areas that are expected to have high variability in signal characteristics and wider spacing in areas that are expected to have more uniform signal characteristics.

In at least one implementation, the scanning device 116 may overlay the measurement region 402 on an artistic rendering of the person 102. For example, the artistic rendering of the person 102 may be specific to anatomical markers within captured images of the person. As a further example, the artistic rendering of the person 102 may reflect the male or female body generally, such as when the gender of the person 102 is input to the scanning device 116 and/or otherwise received or inferred by the scanning device 116. The artistic rendering may be customized based on body type, age group, and/or other demographic factors to provide more accurate visual guidance. The scanning device 116 may use image processing techniques to identify the anatomical markers (e.g., the sternum, the armpit, the collarbone) and automatically adjust the measurement region 402 positioning accordingly. Alternatively, the scanning device 116 may overlay the measurement region 402 on captured images of the person 102. In some implementations, the scanning device 116 may capture real-time images of the measurement area on the person 102 and overlay the measurement region 402 directly onto the live camera feed, providing real-time visual feedback.

The scanning device 116 may determine the measurement locations 408 for measuring the signals (e.g., PPG and/or electrical signals) based on various factors. For instance, the scanning device 116 may receive anatomical data about the person 102, which may be used to determine the placement of the measurement region 402. The anatomical data may include, for example, measurements such as chest circumference, torso length, shoulder width, and/or other dimensional parameters of the person 102. The scanning device 116 may also consider physiological factors such as skin thickness, subcutaneous fat distribution, and muscle mass when determining the measurement locations 408. In at least one implementation, machine learning algorithms may be employed to analyze historical data from similar individuals to predict the measurement locations 408 based on demographic and anatomical characteristics. The scanning device 116 may also incorporate user feedback from previous scanning sessions to refine the measurement location 408 selection process over time. In an additional or alternative example, the scanning device 116 may use image data to determine the measurement locations 408. For instance, the scanning device may use anatomical markers within captured images such as the sternum and armpit to define the region of interest for the measurement area.

In various applications, the measurement locations 408 are influenced by particular physiological parameters of interest and/or characteristics of the signal that are most relevant to those parameters. For instance, applications focused on using PPG measurements to detect heart rate variability may prioritize locations with clear pulsatile waveforms, while those measuring blood oxygen saturation may emphasize areas with good perfusion. Applications monitoring respiratory rate may benefit from measurement locations 408 where the respiratory-induced intensity variations in the signal are more pronounced. The analysis algorithm 120 of the scanning device 116 may adjust the position and/or number of the measurement locations 408 based on the intended application, potentially adjusting a number, spacing, and/or arrangement of the measurement locations 408 to optimize the relevant aspects of the signal for each use case.

Accordingly, once determined, the measurement locations 408 are output in the measurement region 402, and the scanning instructions 406 may prompt the user to begin obtaining the scanning data 118 at the measurement locations 408, as will be further elaborated below.

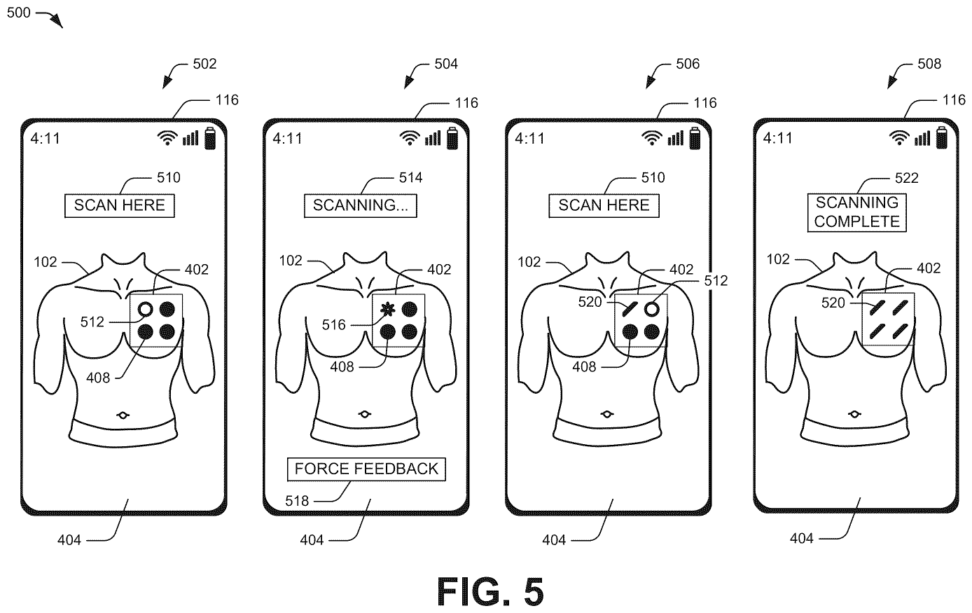

FIG. 5 illustrates a non-limiting example 500 of a sequence of user interfaces output during measurement location scanning by the scanning device 116. The example 500 may be a continuation of the example 400 depicted in FIG. 4, for instance. The example 500 includes a first user interface output 502, a second user interface output 504, a third user interface output 506, and a fourth user interface output 508. It is to be appreciated that there may be additional user interface outputs between one or more or each of the first user interface output 502, the second user interface output 504, the third user interface output 506, and the fourth user interface output 508. The user interface outputs shown in the example 500 may occur at different times during the measurement location scanning, e.g., as a sequence.

In the example 500, the first user interface output 502 includes the scanning device 116 displaying a scan initiation prompt 510 at the top portion of the display screen 404. The scan initiation prompt 510 provides guidance to place the scanning device 116 at one of the measurement locations 408 for an active scan. The scanning device 116 may be configured to read the scan initiation prompt 510 aloud with spoken prompts and/or may produce other forms of auditory and/or non-auditory feedback. The scan initiation prompt 510 may be configured as a pop-up for the user to click through. The scan initiation prompt 510 may be positioned above the measurement region 402, adjacent to the measurement region 402, or below the measurement region 402. The scan initiation prompt 510 may overlay the measurement region 402 to prompt the user to acknowledge the scan initiation prompt 510 before scanning. As a further example, the scan initiation prompt 510 may be configured to disappear after the passage of a predetermined amount of time, e.g., 30 seconds, a minute, or another time duration.

Via the first user interface output 502, the scanning device 116 may guide the user to place the scanning device 116 on the person 102 at a selected location 512 of the measurement locations 408 so that scanning can commence. The first user interface output 502, for example, may represent an initial positioning phase of the scanning device 116. The selected location 512 represents one of the measurement locations 408 that is to be assessed by obtaining the scanning data 118 via the scanning device 116. The scan initiation prompt 510 may include instructions for positioning the scanning device 116 at the selected location 512. For example, the scan initiation prompt 510 may output written instructions, such as “scan here,” “place device here,” or “position scanner at highlighted location.” In at least one implementation, a visual indicator is used to distinguish the selected location 512 from the other measurement locations 408 of the measurement region 402. In the present example, the selected location 512 is shown as a white-filled circle, in contrast to the dark-filled circles representing the other measurement locations 408. Additionally, or alternatively, the scan initiation prompt 510 may provide tactile and/or auditory cues to assist with positioning, such as vibration patterns and/or spoken instructions that guide the user to the selected location 512.

In one or more implementations, the user may be asked to confirm that the scanning device 116 is at the selected location 512 on the person 102 through the scan initiation prompt 510. By way of example, the scan initiation prompt 510 may present a confirmation button or voice prompt asking for confirmation that the scanning device 116 is positioned at the selected location 512. Alternatively, the scanning device 116 may infer that the scanning device 116 is positioned at the selected location 512 after the passage of a predetermined amount of time following the display of the scan initiation prompt 510, e.g., 30 seconds, a minute, or another time duration.

Once the scanning device 116 is positioned at the selected location 512 and confirmed by the user and/or after the predetermined time has elapsed, the scanning device 116 transitions from the positioning phase to an active measurement phase, represented in the second user interface output 504. In the second user interface output 504, the scanning device 116 displays a scan in-progress message 514 at the top portion of the display screen 404. The scan in-progress message 514 communicates to the user that a scan is taking place at an active scan location 516, which corresponds to the selected location 512 shown in the first user interface output 502, for example. The scanning device 116 may be configured to read the scan in-progress message 514 aloud and/or provide another form of auditory, visual, and/or tactile feedback. The scan in-progress message 514 may be positioned above the measurement region 402, adjacent to the measurement region 402, or below the measurement region 402. As a further example, the scan in-progress message 514 may be configured to disappear after the passage of a predetermined amount of time (e.g., five seconds or another predetermined time duration).

Via the scan in-progress message 514, the scanning device 116 may guide the user to maintain the scanning device 116 at the active scan location 516 for at least a threshold amount of time (e.g., five seconds, ten seconds, fifteen seconds, thirty seconds, or another predetermined duration) during which the scanning data 118 is obtained. While the scan is taking place at the active scan location 516, the scanning device 116 may collect the scanning data 118 (e.g., PPG data) by using a light source (e.g., the light source 302) to emit light into tissue at the active scan location 516 and a detector (e.g., the photodetector 304) to capture reflected light from the tissue. By way of example, while positioned at the active scan location 516, the scanning device 116 may continuously sample light intensity variations caused by blood volume changes with each heartbeat and may digitize these optical signals into the scanning data 118. Alternatively, or in addition, the scanning device 116 may collect the scanning data 118 via the one or more additional sensors 314. The scanning device 116 and/or the analysis platform 106 may further process this scanning data 118 (e.g., via the analysis algorithm 120) to extract signal quality metrics such as the signal-to-noise ratio, the pulse amplitude, the consistency at rest, the baseline wander, the perfusion index, the R-wave amplitude, the QRS complex clarity, the baseline stability, the electrode-skin impedance, the P-wave visibility, and/or the T-wave visibility in a real-time or near real-time analysis. Alternatively, the analysis may be performed after scanning is complete for the measurement region 402 (e.g., the scanning data 118 has been collected for all of the measurement locations 408 of the measurement region 402).

In one or more implementations, the duration of measurement at the active scan location 516 may be adjustable based on a desired accuracy, with longer measurement durations generally providing more accurate signal characterization. Moreover, the scanning device 116 may provide real-time feedback during the measurement duration. The real-time feedback may suggest adjustments to application force and/or positioning, for instance, examples of which will be elaborated below. In some implementations, the scanning device 116 may automatically determine (e.g., via the analysis algorithm 120) when a sufficient amount of the scanning data 118 has been obtained at the active scan location 516 and prompt the user to proceed to the next measurement location 408, e.g., via the third user interface output 506.

In the second user interface output 504, the scanning device 116 displays force feedback 518 at the bottom portion of the display screen 404. It will be appreciated, however, that the force feedback 518 can be positioned anywhere on the display screen 404. Alternatively, or in addition, the force feedback 518 may be communicated via auditory and/or tactile feedback. The force feedback 518 may be generated (e.g., by the analysis algorithm 120) based on force data received from the one or more force sensors 312 of the scanning device 116. The force feedback 518 may indicate an amount of force applied by the scanning device 116 (also referred to herein as “applied force”) against the skin of the person 102, e.g., at the active scan location 516. In at least one implementation, the force feedback 518 may further compare the amount of applied force relative to a recommended (e.g., desired) force. Alternatively, or in addition, the force feedback 518 may instruct the user to adjust the amount of force applied by the scanning device 116 during the active measurement phase. For instance, the force feedback 518 may indicate increasing the applied force in response to the applied force being less than the recommended force, indicate decreasing the applied force in response to the applied force being greater than the recommended force, or indicate maintaining the applied force in response to the applied force equaling the recommended force.

The third user interface output 506 shows a navigation aid 520. The navigation aid 520 may, for example, be displayed within the measurement region 402 at the measurement locations 408 that have already been scanned by the scanning device 116. The third user interface output 506 further displays a next measurement location for the selected location 512 and the scan initiation prompt 510. That is, the selected location 512 in the third user interface output 506 is different than the selected location 512 in the first user interface output 502. As an alternative to the navigation aid 520, the absence of a symbol, marker, or icon at a given measurement location within the measurement region 402 may communicate to the user that previously scanned locations do not need to be scanned further. In the example 500, the third user interface output 506 is further configured to display the measurement locations 408 that have not yet been scanned (e.g., as the dark-filled circles).

The scanning device 116 may guide the user through obtaining the scanning data 118 at each (e.g., every) one of the measurement locations 408 in the measurement region 402 until all of the measurement locations 408 have been scanned, resulting in the fourth user interface output 508. The fourth user interface output 508 illustrates one example of the scanning device 116 after the scanning device 116 has finished collecting the scanning data 118 to determine sensor placement. In the example shown by the fourth user interface output 508, the scanning device 116 displays a scanning completion message 522, and the navigation aid 520 is displayed at each of the measurement locations 408 within the measurement region 402. The scanning completion message 522 communicates the end of the location scanning process, such as when there are no further measurement locations 408 to be assessed by the scanning device 116 during a current scanning process. The scanning device 116 may be configured to read the scanning completion message 522 aloud and/or may provide another form of visual, auditory, and/or tactile feedback that indicates the scanning process has been completed. The scanning completion message 522 may be configured as a pop-up for the user to click through. The scanning completion message 522 may be positioned above the measurement region 402, below the measurement region 402, or adjacent to the measurement region 402. The scanning completion message 522 may overlay the measurement region 402 to indicate the end of the scanning process. As a further example, the scanning completion message 522 may be configured to disappear after the passage of a predetermined amount of time, e.g., 30 seconds, a minute, or another time duration.

In at least one example, the scanning data 118 obtained throughout the example 500 is transmitted to the analysis platform 106 while the scanning data 118 is being obtained and/or after completion of the scanning process. The prediction system 114 of the analysis platform 106 may analyze the scanning data 118 (e.g., via the analysis algorithm 120) to determine a recommended location for sensor placement, which may be communicated back to the scanning device 116 as the prediction 110 in one or more implementations. Additionally, or alternatively, the scanning device 116 processes the scanning data 118 locally.

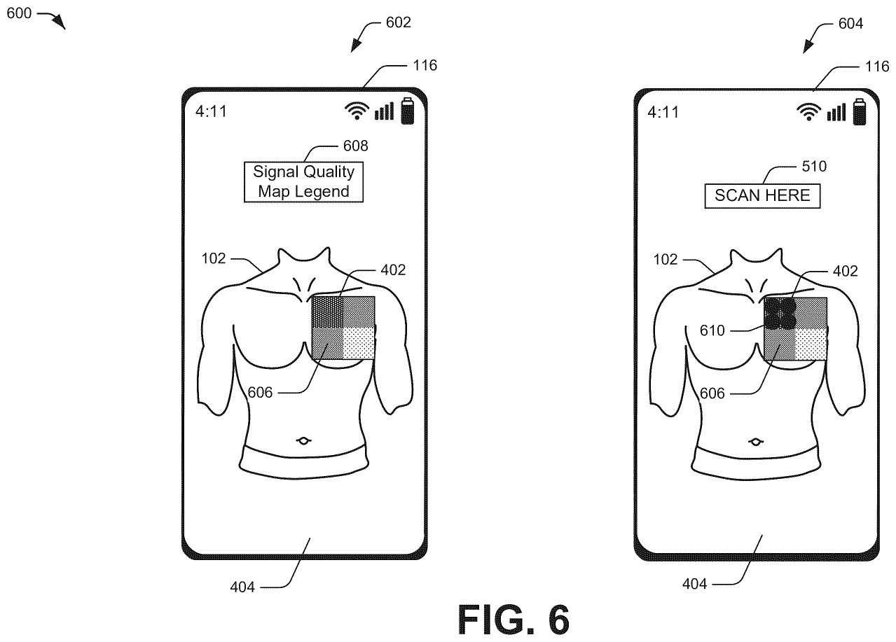

FIG. 6 illustrates a non-limiting example 600 of a sequence of user interfaces output by the scanning device 116 during analysis of the scanning data 118. The example 600 may be a continuation of the example 500 depicted in FIG. 5, for example. The example 600 includes a fifth user interface output 602 and a sixth user interface output 604. It is to be appreciated that there may be additional interface outputs between the fifth user interface output 602 and the sixth user interface output 604. The interface outputs in the example 600 may occur at different times during the analysis of the scanning data 118, e.g., as a sequence. In at least one implementation, a user may access the interface outputs in the example 600 (e.g., by interacting with controls displayed by the display screen 404).

In the example 600, the fifth user interface output 602 displays a signal quality map 606 of the measurement region 402 on the display screen 404 along with a signal quality map legend 608. The signal quality map 606 may be generated by the analysis algorithm 120 based on the scanning data 118 obtained at the measurement locations 408 and may provide a visual representation of at least one signal quality and/or measurement characteristic of the scanning data 118. In an implementation where PPG sensor placement is being determined, the signal quality map 606 may be a perfusion map of blood flow characteristics and/or PPG signal quality characteristics across the measurement region 402, indicating variations in tissue perfusion levels at different locations. The signal quality map legend 608 may indicate how visual elements of the signal quality map 606 correspond to perfusion and/or signal quality characteristics. For example, the signal quality map 606 may include colors, patterns, shading, and/or symbols to indicate aspects of the scanning data 118 (e.g., areas of higher perfusion versus areas of lower perfusion, areas of higher signal quality versus areas of lower signal quality, or the like), and the signal quality map legend 608 may provide a key for interpreting the visual elements of the signal quality map 606. In some implementations, the signal quality map legend 608 may include a continuous gradient to represent a range of values between a minimum and a maximum. Alternatively, the signal quality map legend 608 may use discrete categories or ranges to represent different perfusion levels and/or deviations from a desired perfusion level (or signal quality). For example, the signal quality map legend 608 may communicate areas of the signal quality map 606 that correspond to areas of the person 102 exhibiting high perfusion (and/or high signal quality) and areas of the signal quality map 606 that correspond to areas of the person 102 exhibiting low perfusion (and/or low signal quality).

In one or more implementations, via the analysis algorithm 120 of the prediction system 114, the analysis platform 106 may compute various metrics for each measurement location 408, such as pulse amplitude, pulse rate, a consistency at rest, a baseline wander, and/or perfusion index (which may be calculated as the ratio of pulsatile to non-pulsatile signal components) for PPG measurements, and/or characteristics of the electrical signals such as R-wave amplitude, signal-to-noise ratio, QRS complex clarity, baseline stability, electrode-skin impedance, and/or P-wave and T-wave visibility. The prediction system 114 may perform normalization and/or mapping to convert these metrics into the signal quality map 606. In at least some implementations, the signal quality map 606 may be output as a color-coded visualization, e.g., a heat map. It is to be appreciated, however, that in at least one variation, the signal quality map 606 is not output for display on the display screen 404 but is still usable by the analysis platform 106 to evaluate the scanning data 118.

In the example 600, the signal quality map 606 is displayed on the display screen 404 of the scanning device 116 as a two-dimensional map overlaid on the measurement region 402. The signal quality map 606 in this example includes darker pattern shadings to indicate higher perfusion signals and lighter pattern shadings to indicate lower perfusion signals. Areas on the person 102 with higher perfusion signals may correspond to areas on the person 102 exhibiting stronger arterial flow and/or greater signal quality. Areas on the person 102 with lower perfusion signals may correspond to weaker arterial flow and/or reduced signal quality.

In the example 600, the sixth user interface output 604 includes the scan initiation prompt 510 at the top of the display screen 404 and additional measurement locations 610. By way of example, based on the signal quality map 606, the sixth user interface output 604 indicates that the scanning data 118 is to be obtained at the additional measurement locations 610, which may be positioned differently than the measurement locations 408 and/or may overlap with the measurement locations 408. The sixth user interface output 604 depicts four additional measurement locations 610 overlaid on the signal quality map 606. It will be appreciated, however, that any number of additional measurement locations 610 may be determined by the analysis algorithm 120 and displayed by the sixth user interface output 604.

In some instances, at least a portion of the additional measurement locations 610 correspond to one or more locations that are already associated with scanning data 118, e.g., to obtain a repeated measurement. In some instances, repeating measurements at the same location can confirm the accuracy of an earlier recorded measurement. Repeating measurements at the same location may provide multiple measurements for performing a statistical analysis, e.g., determining an average. Alternatively, or in addition, the additional measurement locations 610 may correspond to locations that are not yet associated with scanning data 118. By way of example, the analysis algorithm 120 may determine the additional measurement locations 610 to refine the signal quality map 606. At least a portion of the additional measurement locations 610 may be positioned between two or more of the measurement locations 408 to provide finer resolution in areas showing higher perfusion and/or signal quality characteristics, for instance. The additional measurement locations 610 may be part of an iterative scanning approach to identify a true maximum signal quality location rather than a local maximum. For instance, the measurement locations 408 may provide an initial coarse mapping, and the additional measurement locations 610 may provide a targeted refinement to determine the location with the best overall signal characteristics across the measurement region 402 for the specific application. It will be appreciated that in at least one variation, one or more of the additional measurement locations 610 may be outside of the measurement region 402.

In one or more implementations, the analysis algorithm 120 may estimate a position within the measurement region 402 that has the highest signal quality by identifying areas in the signal quality map 606 where the quality metrics are highest. For example, the analysis algorithm 120 may identify an area of the signal quality map 606 that exhibits higher quality values relative to surrounding areas and estimate that the position with the highest quality lies within or near this area. The analysis algorithm 120 may apply interpolation techniques to the signal quality map 606 to estimate signal quality values at positions between the measurement locations 408. Based on the interpolated values, the analysis algorithm 120 may estimate the specific position within the measurement region 402 that is expected to have the highest signal quality. The additional measurement locations 610 may then be positioned at and/or around the estimated position to verify and refine the estimation through direct measurement.

As mentioned above, the analysis algorithm 120 may implement an iterative refinement process to identify a recommended location for placement of the monitoring device 104. For example, after obtaining the scanning data 118 at the additional measurement locations 610, the analysis algorithm 120 may update the signal quality map 606 to incorporate the newly obtained scanning data 118. The updated signal quality map 606 may provide increased resolution and/or accuracy at the additional measurement locations 610. Based on the updated signal quality map 606, the analysis algorithm 120 may determine whether to request further additional measurement location(s) to refine the determination of the recommended location. In some implementations, the analysis algorithm 120 may continue to request the scanning data 118 from additional measurement location(s) and update the signal quality map 606 until a convergence condition is satisfied. The convergence condition may include determining that a location with the highest signal quality has been identified with sufficient confidence, determining that additional measurements would not substantially improve the accuracy of the recommended location, and/or determining that a predetermined number of iterations has been completed. Alternatively, or in addition, the convergence condition may include determining that a signal quality metric at a particular location exceeds a threshold value and/or determining that a difference in signal quality between successive iterations is below a threshold difference.

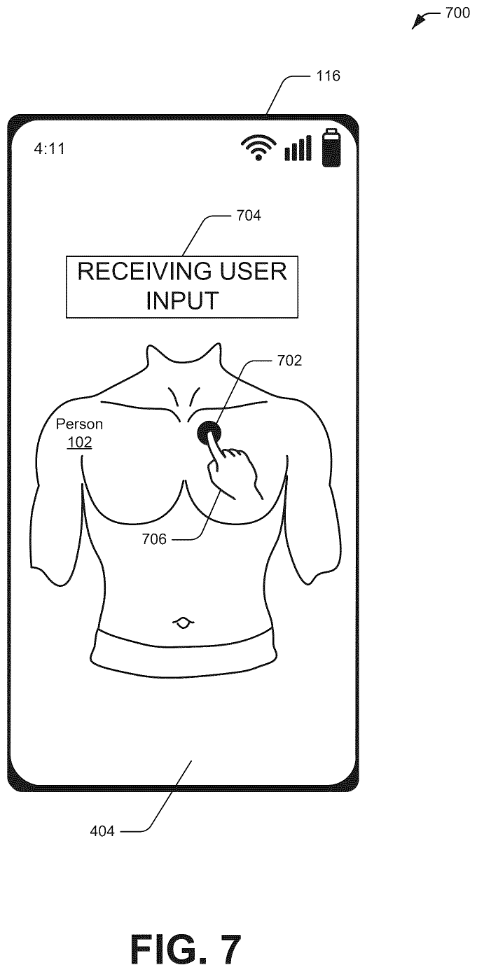

FIG. 7 illustrates a non-limiting example 700 of the scanning device 116 receiving user input. The example 700 of FIG. 7 may be used in conjunction with the example 400 of FIG. 4, the example 500 of FIG. 5, and/or the example 600 of FIG. 6.

The example 700 illustrates the scanning device 116 receiving a selection of at least one user-selected measurement location 702 from the user. To do so, the scanning device 116 displays a user input prompt 704 at the top portion of the display screen 404. The user input prompt 704 requests input from the user to manually select the at least one user-selected measurement location 702 for scanning. In the example 700, the user manually selects the at least one user-selected measurement location 702 using a location selector 706. By way of example, the location selector 706 may be an interactive element that the user can move around the display screen 404 (e.g., by touch, control buttons, voice control, or another type of input) to establish where the scanning data 118 is to be obtained. As an example, the display screen 404 may be a touch screen configured to allow the user to move the location selector 706 through physical contact with the display screen 404. Alternatively, or additionally, the display screen 404 may include control buttons with commands to move the location selector 706 up, down, or sideways. In some instances, the user may move the location selector 706 to the at least one user-selected measurement location 702 before performing a scan at the at least one user-selected measurement location 702. Alternatively, the user may move the location selector 706 to the at least one user-selected measurement location 702 after performing a scan at the at least one user-selected measurement location 702.

In at least one implementation, the scanning device 116 may recommend a predetermined number of measurement locations for the user to select via the location selector 706. As a non-limiting example, the scanning device 116 may recommend a number of locations in a range from one to thirty, such as four locations, nine locations, sixteen locations, twenty-five locations, or another number of locations. Alternatively, the user may determine the number of locations to select via the location selector 706. The scanning data 118 may be obtained at the at least one user-selected measurement location 702 in addition to the measurement locations 408 and/or the additional measurement locations 610.

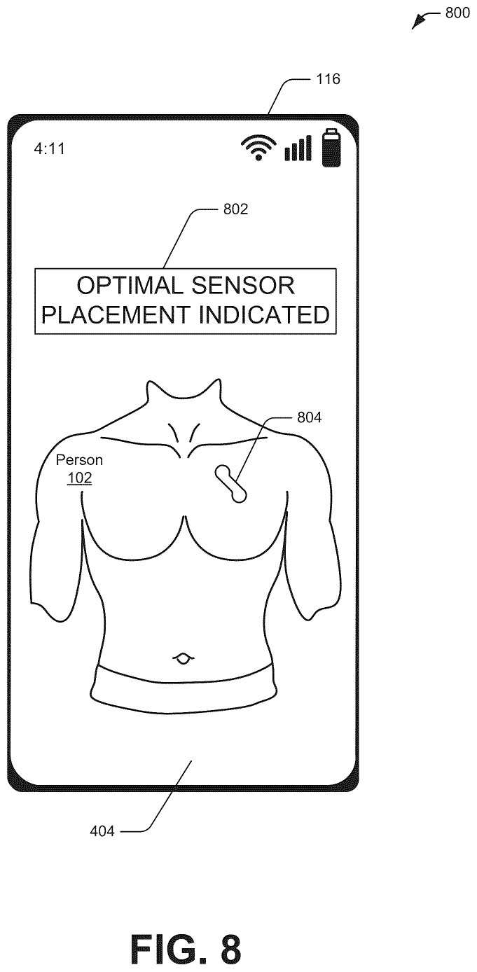

FIG. 8 illustrates a non-limiting example 800 of a result of the scanning process using the scanning device 116. The example 800 may be a continuation of the example 400 depicted in FIG. 4, the example 500 depicted in FIG. 5, the example 600 depicted in FIG. 6, and/or the example 700 depicted in FIG. 7. The example 800 illustrates one example of how sensor placement is indicated after analysis of the scanning data 118 (e.g., the PPG signals and/or electrical signals) obtained during the scanning process.

In the example 800, the scanning device 116 determines a sensor placement in accordance with the techniques described herein and displays a placement indication 802 at the top portion of the screen. In the illustrated example, the placement indication 802 includes text that reads “OPTIMAL SENSOR PLACEMENT INDICATED,” although the placement indication 802 includes other messages in variations. Moreover, in at least one variation, the placement indication 802 is output via auditory and/or tactile feedback in addition to or as an alternative to a visual message. A recommended location 804 is depicted on a representation of the person 102, indicating the location for placement of the monitoring device 104, and thus the sensor (e.g., the PPG sensor or electrical sensor).

In at least one example, the recommended location 804 is determined based on an analysis of the scanning data 118 obtained during the scanning process, e.g., via the analysis algorithm 120, such as described herein. In various implementations, the analysis platform 106 generates, as the one or more predictions 110, the recommended location 804 based on the signal quality map 606, which is communicated to the scanning device 116 for presentation to the user. By way of example, the analysis platform 106 may determine the recommended location 804 by analyzing the signal quality map 606 to identify suitable signal quality and/or perfusion characteristics across the measurement region 402. The analysis platform 106 may compare various metrics across the measurement region 402, such as the signal-to-noise ratio, the pulse amplitude, and the perfusion index values for PPG measurements, and/or characteristics of the electrical signals such as R-wave amplitude, signal-to-noise ratio, QRS complex clarity, baseline stability, electrode-skin impedance, and/or P-wave and T-wave visibility. In one or more implementations, the analysis algorithm 120 may apply selection criteria to identify locations with enhanced signal quality, which may include areas with strong pulsatile signals, high perfusion index values, and/or minimal noise interference. When PPG sensor placement is being determined, the analysis algorithm 120 may further evaluate the spatial distribution of perfusion characteristics to determine locations that provide consistent and reliable PPG signal acquisition. The analysis algorithm 120 may also interpolate or otherwise analyze areas that were not directly scanned to determine the recommended location 804. In some examples, the selection process performed by the analysis algorithm 120 may include ranking the measurement locations 408, the additional measurement locations 610, the at least one user-selected measurement location 702, and/or an interpolated location based on one or more quality metrics (e.g., which may be or may be derived from the signal-to-noise ratio, the pulse amplitude, the perfusion index values, the R-wave amplitude, the QRS complex clarity, the consistency at rest, the baseline stability or wander, the electrode-skin impedance, the P-wave visibility, and/or the T-wave visibility) and selecting the location with the highest overall score. The analysis algorithm 120 may also consider factors such as signal stability over time and resistance to motion artifacts when determining the recommended location 804, such as when the scanning data 118 have been acquired at multiple different time points.

The following discussion describes techniques that are implementable utilizing the previously described systems and devices. Aspects of the procedure (e.g., method) can be implemented in hardware, firmware, software, or a combination thereof. The procedure is shown as a set of blocks that specify operations that can be performed by one or more devices and are not necessarily limited to the orders shown for performing the operations by the respective blocks. One or more blocks of the procedure, for instance, specify operations that can be programmable by hardware (e.g., a processor, microprocessor, controller, and/or firmware) as executable instructions, thereby creating a special purpose machine for carrying out an algorithm as illustrated by the flow diagram. As a result, the instructions are storable on a computer-readable storage medium that causes the hardware to perform the algorithm. In portions of the following discussion, reference will be made to FIGS. 1-8.

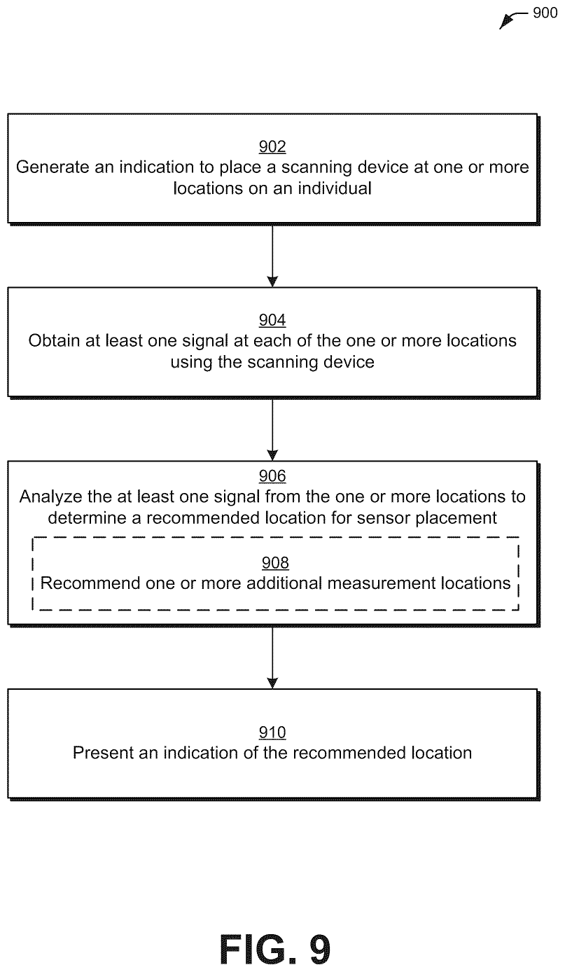

FIG. 9 illustrates a flowchart of a method 900 for determining a location for placement of a sensor, such as a PPG sensor or an ECG electrode. The method 900 includes a sequence of steps for scanning and analyzing different chest locations to identify a recommended sensor placement position.

An indication to place a scanning device at one or more locations on an individual is generated (block 902). For instance, instructions are displayed by the scanning device 116 to guide the user in positioning the scanning device 116 at the measurement locations 408 on a chest region of the person 102. In a non-limiting example, the instructions include the scan initiation prompt 510 of example 500. The scan initiation prompt 510 may guide the user to place the scanning device 116 at one of the measurement locations 408 for an active scan. The measurement locations 408 may be displayed by the display screen 404 of the scanning device 116 within the measurement region 402 on the person 102. The indication to place the scanning device at the one or more measurement locations may include indicating the selected location 512 from among the measurement locations 408. The selected location 512 corresponds to the location at which the scanning device 116 is to be currently placed for obtaining a signal (e.g., a PPG signal). The user may be asked to confirm that the scanning device 116 is at the selected location 512 before the scanning device 116 obtains the scanning data 118. Additionally, or alternatively, the user can manually select at least a portion of the one or more locations (e.g., as the at least one user-selected measurement location 702), such as according to the example 700 of FIG. 7.

At least one signal is obtained at each of the one or more locations using the scanning device (block 904). By way of example, the scanning device 116 is configured to measure a PPG signal at each of the measurement locations 408 by using the light source 302 to transmit light into the person 102 and the photodetector 304 to detect light reflected from the person 102. The PPG signal measured at each of the measurement locations 408 comprises the scanning data 118. In some cases, measuring the PPG signal may include measuring at least one of a signal-to-noise ratio, a pulse amplitude, a consistency at rest, a baseline wander, or a perfusion index. In one or more implementations, the scanning device 116 may be configured to obtain other types of signals, such as electrical signals (e.g., ECG and/or impedance measurements), depending on the use case and the type of sensor for which sensor placement is being determined. The scanning device 116 may include the one or more additional sensors 314 to capture such alternative or additional signals. When the scanning device 116 is implemented as a smartphone, the one or more additional sensors 314 may be included in the accessory 316 that communicates with the smartphone via wired or wireless communication protocols.

In a non-limiting example, the display screen 404 of the scanning device 116 displays the scan in-progress message 514, which communicates to the user that the scanning data 118 is being acquired. Additionally, the measurement region 402 may display the active scan location 516 corresponding to the position within the measurement region 402 where the at least one signal is being obtained by the scanning device 116.

The scanning device 116 may further output the force feedback 518 regarding the amount of force applied by the scanning device 116 to the skin of the person 102 based on force data from the one or more force sensors 312. The force feedback 518 may include instructions to increase the applied force (e.g., in response to the applied force being less than the desired force for obtaining the scanning data 118), decrease the applied force (e.g., in response to the applied force being greater than the desired force for obtaining the scanning data 118), or maintain the applied force (e.g., in response to the applied force being equal to the desired force for obtaining the scanning data 118). The force feedback 518 may be provided as visual feedback, tactile feedback, or auditory feedback. In some implementations, the force feedback 518 may include numerical indicators, color-coded indicators, haptic vibrations, and/or spoken prompts.

The at least one signal from the one or more locations is analyzed to determine a recommended location for sensor placement (block 906). For instance, the scanning device 116 may implement one or more algorithms (e.g., the analysis algorithm 120) to determine the recommended location 804 based on the scanning data 118. When PPG sensor placement is being determined, the analysis algorithm 120 may analyze various characteristics of the PPG signals, which may include the signal-to-noise ratio, the pulse amplitude, the perfusion index, the waveform morphology, the pulse peak strength, and/or the signal stability. In at least one example, the recommended location 804 is generated based on the signal quality map 606 by the analysis algorithm 120 computing metrics for each location and performing normalization and mapping to convert the metrics into a representation that indicates blood flow characteristics across the measurement region 402. In a non-limiting example, the display screen 404 of the scanning device 116 displays the signal quality map 606 along with the signal quality map legend 608, which communicates how to interpret the signal quality map 606. The signal quality map 606 may use colors, patterns, shading, or symbols to indicate areas of higher perfusion versus areas of lower perfusion, for example, as indicated by the signal quality map legend 608.

In one or more implementations where ECG sensor placement is being determined, the analysis algorithm 120 may analyze characteristics of electrical signals obtained by the one or more additional sensors 314, such as ECG signals and/or impedance measurements. For example, when determining optimal placement for an ECG sensor, the analysis algorithm 120 may analyze characteristics of the electrical signals such as R-wave amplitude, signal-to-noise ratio, QRS complex clarity, baseline stability, electrode-skin impedance, and/or P-wave and T-wave visibility. The analysis algorithm 120 may generate the signal quality map 606 based on the electrical signal characteristics obtained at the measurement locations 408, where the signal quality map 606 indicates variations in electrical signal quality across the measurement region 402. The recommended location 804 may be determined based on the signal quality map by identifying locations that exhibit enhanced electrical signal characteristics for the intended application of the monitoring device 104.

In one or more implementations, the scanning device 116 may include multiple different sensor types to obtain multiple different signal types at the measurement locations 408. For example, the scanning device 116 may obtain both PPG signals and electrical signals (e.g., ECG signals) at each of the measurement locations 408. The analysis algorithm 120 may generate a signal quality map for each signal type, such as a perfusion map based on the PPG signals and an electrical signal quality map based on the ECG signals. The analysis algorithm 120 may determine the recommended location 804 for placement of a multi-sensor monitoring device by balancing signal quality characteristics across the different signal types. For instance, the analysis algorithm 120 may identify a location that provides acceptable signal quality for both PPG and ECG measurements, even if that location does not represent the highest signal quality for either signal type individually. The analysis algorithm 120 may apply weighting factors to the different signal types based on the intended application of the monitoring device 104, such that signal types of greater importance to the application are weighted more heavily when determining the recommended location 804. In some implementations, the analysis platform 106 may present multiple candidate locations to the user along with signal quality metrics for each signal type at each candidate location, allowing the user to select a location based on the relative importance of the different signal types for the intended use case.

Optionally, one or more additional measurement locations are recommended (block 908). By way of example, based on the signal quality map 606, the analysis algorithm 120 of the analysis platform 106 may recommend the additional measurement locations 610. The additional measurement locations 610 may be positioned between two or more of the measurement locations 408 to provide finer resolution in areas showing higher perfusion and/or signal quality characteristics. The additional measurement locations 610 recommended by the analysis algorithm 120 may be displayed by the display screen 404 along with the scan initiation prompt 510. The analysis algorithm 120, for instance, may implement an iterative refinement process whereby the scanning data 118 is obtained at the additional measurement locations 610, the signal quality map 606 is updated to incorporate the newly obtained scanning data 118, and any further additional measurement locations are determined based on the updated signal quality map 606. The analysis algorithm 120 may continue to output prompts for additional measurement locations and update the signal quality map 606 until a convergence condition is satisfied. The convergence condition may include determining that a location with a highest signal quality has been identified with sufficient confidence, determining that additional measurements are not predicted to substantially improve the accuracy of the recommended location 804, determining that a predetermined number of iterations has been completed, determining that a signal quality metric at a particular location exceeds a threshold value, and/or determining that the difference in signal quality between successive iterations is less than a threshold difference.

An indication of the recommended location is presented (block 910). For instance, the indication is output in a user interface. The indication may guide the user to place the monitoring device 104 or the one or more sensors 202 of the monitoring device 104 at the determined location for subsequent measurement based on one or more qualities of the signals of the scanning data 118. In at least one example, the display screen 404 of the scanning device 116 displays the placement indication 802, thereby instructing the user to place the monitoring device 104 at the recommended location 804. The recommended location 804 may be displayed by the display screen 404 of the scanning device 116. In at least one example, the skin-facing surface 310 of the scanning device 116 includes the physical alignment aid 308 that is configured to mark the recommended location 804 on the skin of the person 102. By way of example, the physical alignment aid 308 may create a temporary indent in the skin when the scanning device 116 is pressed against the skin at the recommended location 804. Placement of the sensor may then be based on the mark left by the scanning device 116, which may help ensure consistent and accurate positioning of the monitoring device 104 at the location determined to provide optimal signal quality.

By following the method 900, the scanning device 116 and the analysis platform 106 work together to determine an optimal location for sensor (e.g., PPG sensor) placement. By way of example, the approach of the method 900 may improve a quality and reliability of PPG measurements taken by the monitoring device 104 by accounting for individual anatomical variations of the person 102 and identifying locations with enhanced signal quality and/or perfusion characteristics. The method 900 may enable personalized sensor placement that enhances signal quality for each individual, which may improve the accuracy of physiological monitoring while reducing motion artifacts and/or other sources of signal degradation.

### Machine Learning and AI in Scanning Techniques to Determine Sensor Placement

The previous examples describe various instances of artificial intelligence (“AI”) models and/or machine learning models such as with respect to the analysis algorithm 120 and/or the prediction system 114. In one or more examples, an AI model, e.g., a machine learning model, refers to a computer representation that is tunable (e.g., through training and retraining) based on inputs without being actively programmed by a user to approximate unknown functions, automatically and without user intervention. For instance, the term machine learning model includes a model that utilizes algorithms to learn from, and make predictions on, known data by analyzing training data to learn and relearn to generate outputs that reflect patterns and attributes of the training data.

In the context of determining sensor placement locations, machine learning models are implementable (e.g., by one or more processing devices of the analysis platform 106 and/or the scanning device 116) to analyze signal quality data patterns, such as to identify recommended placement locations and generate the signal quality map 606. For example, the analysis algorithm 120 and/or the prediction system 114 may each utilize one or more machine learning models to process physiological signal data such as PPG signals, electrical signals, perfusion characteristics, and/or other measurements collected by the scanning device 116. Examples of machine learning models applicable to sensor placement determination include neural networks, convolutional neural networks (CNNs) such as for analyzing waveform data and signal quality patterns, long short-term memory (LSTM) neural networks such as to analyze temporal signal characteristics, generative adversarial networks (GANs), decision trees (e.g., for location classification), support vector machines, linear regression, logistic regression for binary quality assessments, Bayesian networks, random forest learning for feature importance in signal data, dimensionality reduction algorithms, boosting algorithms, deep learning neural networks, and so forth.

A machine learning model, for instance, is configurable using a plurality of layers having, respectively, a plurality of nodes. The plurality of layers is configurable to include an input layer, an output layer, and one or more hidden layers. In the context of sensor placement determination, the input layer may receive various signal quality parameters from the scanning data 118, such as signal-to-noise ratio values, pulse amplitude measurements, perfusion index values, consistency at rest metrics, baseline wander characteristics, waveform characteristics or features, and so forth. The hidden layers, for instance, process these inputs through weighted connections to identify complex patterns indicative of suitable sensor placement locations, e.g., patterns that are not detectable using conventional analysis modalities. The output layer may produce the recommended location 804 indicating a placement position for the monitoring device 104 and/or generate the signal quality map 606 representing perfusion and/or signal quality characteristics across the measurement region 402. Calculations are performed by the nodes within the layers via hidden states through a system of weighted connections that are “learned” during training of the machine learning model to implement a variety of sensor placement assessment tasks.

In order to train the machine learning model for sensor placement determination, training data are received that provide examples of “what is to be learned” by the machine learning model, i.e., as a basis to learn patterns from the data. For sensor placement applications, the training data may include labeled datasets of signal quality measurements from individuals with known anatomical characteristics (e.g., height, weight, BMI, gender) and corresponding placement locations that yielded high-quality signals. A machine learning system that includes the machine learning model, for instance, collects and preprocesses the training data that include input features (e.g., PPG waveforms, signal-to-noise ratio values, perfusion index measurements, anatomical data about the individual) and corresponding target labels (e.g., “high signal quality location,” “low signal quality location,” “recommended placement position,” or specific coordinates within the measurement region 402).

The machine learning system is further operable to initialize various parameters of the machine learning model, which are usable by the machine learning model as internal variables to represent and process information during training. These parameters are further usable to represent inferences gained through training. In one or more implementations, the training data are separated into batches to improve processing and optimization efficiency of the parameters of the machine learning model during training, which may be beneficial for model accuracy when processing signal quality data from multiple measurement locations 408 across diverse anatomical variations.

The training data are then received by the machine learning model as inputs and used to generate predictions based on a current state of parameters of layers and corresponding nodes of the model, a result of which is output as output data, e.g., the recommended location 804, the signal quality map 606, the additional measurement locations 610, etc. For example, the analysis platform 106 includes a machine learning model that is trained to recognize patterns in signal quality data that correlate with suitable sensor placement positions, which enables the analysis platform 106 to generate accurate placement recommendations and provide the signal quality map 606 that guides iterative refinement of the scanning process.

Training of the machine learning model can include calculation of a loss function to quantify a loss associated with operations performed by nodes of the machine learning model. The loss function is configurable in various ways to control operation and/or functionality of the machine learning model. For instance, the loss function may be designed to prioritize accuracy in identification of high signal quality locations while minimizing recommendations that could lead to suboptimal sensor placement. Calculation of the loss function, for instance, includes comparing a difference between predictions specified in the output data (e.g., predicted placement locations or signal quality assessments) with target labels specified by the training data (e.g., verified high-quality placement positions). The loss function is configurable in a variety of ways, examples of which include regret, a quadratic loss function as part of a least squares technique for continuous signal quality parameters, cross-entropy loss for location classification tasks, custom loss functions that incorporate anatomical factors specific to particular body regions, and so forth.

The training data are usable to support a variety of usage scenarios in sensor placement determination. For example, the machine learning model can be trained to detect specific patterns in PPG data that indicate areas of high perfusion, identify signal characteristics indicative of suitable electrode placement for ECG measurements, recognize anatomical variations that affect signal quality across different individuals, or predict placement locations based on anatomical data about the individual without requiring exhaustive scanning of the measurement region 402. The models can be configured to operate within computational constraints of the scanning device 116 while providing accurate placement recommendations. The models can further be reconfigured, e.g., with expanded capabilities, for a relatively more resource-intensive analysis when warranted by complex anatomical variations or when determining placement for multi-sensor monitoring devices. This adaptive approach enables efficient use of computational resources devoted to machine learning processes while ensuring comprehensive analysis is available when needed, all using the scanning data 118 collected during the scanning process.

It should be understood that many variations are possible based on the disclosure herein. Although features and elements are described above in particular combinations, each feature or element is usable alone without the other features and elements or in various combinations with or without other features and elements.

What is claimed is:

1. A method for determining a location for placement of a monitoring device, comprising:
generating an indication to place a scanning device at one or more measurement locations within a measurement region on an individual;
obtaining, by the scanning device, a signal at each of the one or more measurement locations;
analyzing the signal obtained at the one or more measurement locations to determine a recommended location for the placement of the monitoring device; and
outputting the recommended location.

2. The method of claim 1, further comprising:
receiving anatomical data about the individual; and
determining the one or more measurement locations based in part on the anatomical data.

3. The method of claim 1, wherein the measurement region is at least a portion of a chest region of the individual.

4. The method of claim 1, wherein obtaining, by the scanning device, the signal at each of the one or more measurement locations includes contacting the scanning device with the individual at each of the one or more measurement locations, and the method further comprises:
measuring, at the one or more measurement locations and via at least one force sensor of the scanning device, a force of the scanning device against the individual;
generating force feedback for pressing the scanning device against the individual based on the measured force relative to a recommended force; and
presenting the force feedback via the scanning device.

5. The method of claim 4, wherein the force feedback includes one or more of visual feedback, haptic feedback, or auditory feedback.

6. The method of claim 1, further comprising:
determining a quality of the signal obtained at the one or more measurement locations; and
outputting a prompt to place the scanning device at one or more additional measurement locations within the measurement region based on the quality of the signal obtained at the one or more measurement locations.

7. The method of claim 6, wherein the quality includes at least one of a signal-to-noise ratio, a pulse amplitude, a perfusion index, a consistency at rest, or a baseline wander, and wherein outputting the prompt to place the scanning device at the one or more additional measurement locations based on the quality of the signal obtained at the one or more measurement locations comprises:
generating a signal quality map by mapping the quality of the signal obtained at the one or more measurement locations to the measurement region of the individual;
estimating a position within the measurement region that has a highest quality of the signal based on the signal quality map; and
outputting the prompt to place the scanning device at the one or more additional measurement locations within the measurement region based on the estimated position.

8. The method of claim 1, wherein the scanning device is configured to mark the individual at the recommended location.

9. The method of claim 1, wherein the signal is a photoplethysmography (PPG) signal, and the scanning device comprises a smartphone with a light source configured to emit light toward skin of the individual and a photodetector configured to measure reflected light from the skin to obtain the PPG signal.

10. A system for determining a placement location of a monitoring device, comprising:
a scanning device having at least one sensor configured to obtain at least one physiological signal from an individual;
at least one processor configured to:
generate an indication to place the scanning device at one or more measurement locations within a measurement region on the individual;
receive the at least one physiological signal obtained by the scanning device at the one or more measurement locations; and
generate a recommended location for the placement location of the monitoring device within the measurement region based on the at least one physiological signal obtained at the one or more measurement locations; and
a display configured to present the recommended location.

11. The system of claim 10, wherein the at least one sensor includes a photoplethysmography (PPG) sensor configured to obtain a PPG signal from the individual, the PPG sensor comprising:
a light source configured to emit light toward skin of the individual; and
a photodetector configured to detect reflected light from the skin.

12. The system of claim 10, wherein the at least one sensor includes an electrode configured to obtain an electrical signal from the individual.

13. The system of claim 10, wherein the at least one sensor includes both of a PPG sensor configured to obtain a PPG signal from the individual and an electrode configured to obtain an electrical signal from the individual.

14. The system of claim 10, wherein the at least one processor is further configured to:
determine a signal quality of the at least one physiological signal obtained at the one or more measurement locations;
generate at least one signal quality map based on the signal quality of the at least one physiological signal obtained at the one or more measurement locations; and
generate the recommended location for the placement location of the monitoring device based on the at least one signal quality map.

15. The system of claim 14, wherein the signal quality includes at least one of a signal-to-noise ratio, an amplitude, a perfusion index, a consistency at rest, a baseline wander, a QRS complex clarity, a baseline stability, an electrode-skin impedance, a P-wave visibility, or a T-wave visibility.

16. The system of claim 10, wherein the scanning device further comprises:
at least one force sensor positioned on a skin-facing surface of the scanning device, the at least one force sensor configured to measure an amount of force applied to skin of the individual by the scanning device; and
at least one physical alignment on the skin-facing surface and configured to mark the individual at the recommended location when contacted with the skin of the individual.

17. A method for determining a location for placement of a monitoring device having at least one sensor, comprising:
generating, via a scanning device, an indication to place the scanning device at one or more measurement locations within a measurement region on an individual;
obtaining, via the scanning device, at least one signal at each of the one or more measurement locations;
generating, by an analysis platform, a signal quality map of the measurement region based on the at least one signal at each one of the one or more measurement locations;
determining, by the analysis platform, a recommended location for the placement of the monitoring device based on the signal quality map; and
indicating the recommended location via the scanning device.

18. The method of claim 17, further comprising:
receiving anatomical data about the individual; and
determining the one or more measurement locations based at least in part on the anatomical data.

19. The method of claim 17, wherein the measurement region is within a chest region of the individual.

20. The method of claim 17, wherein the at least one signal includes both of a photoplethysmography (PPG) signal and an electrical signal, and determining, by the analysis platform, the recommended location for the placement of the monitoring device based on the signal quality map comprises:
generating a perfusion map based on the PPG signal;
generating an electrical signal quality map based on the electrical signal; and
applying weighting factors to the perfusion map and the electrical signal quality map based on an intended application of the monitoring device to determine the recommended location.
